(19) United States
(12) Patent Application Publication
(10) Pub. No.: US 2021/0002677 A1
(43) Pub. Date: Jan. 7, 2021

(54) SYNTHESIS OF OMEGA FUNCTIONALIZED PRODUCTS

(71) Applicant: Ramon GONZALEZ, Tampa, FL (US)

(72) Inventors: Ramon GONZALEZ, Tampa, FL (US); Seokjung CHEONG, Emeryville, CA (US); James M. CLOMBURG, Tampa, FL (US)

(21) Appl. No.: 17/021,338

(22) Filed: Sep. 15, 2020

Related U.S. Application Data
(63) Continuation-in-part of application No. 15/784,691, filed on Oct. 16, 2017.
Application No. 15/784,691 is a continuation of application No. PCT/US2016/029592, filed on Apr. 27, 2016.
Application No. 15/784,691 is a continuation of application No. PCT/US16/29583, filed on Apr. 27, 2016.
Application No. 15/784,691 is a continuation of application No. PCT/US16/27903, filed on Apr. 15, 2016.
(60) Provisional application No. 62/154,397, filed on Apr. 29, 2015.
(60) Provisional application No. 62/154,010, filed on Apr. 28, 2015.
(60) Provisional application No. 62/148,248, filed on Apr. 16, 2015.

(51) Int. Cl. C12P 7/42; C12P 5/02; C12P 7/16; C12N 1/20

(52) U.S. Cl. CPC C12P 7/42; C12P 5/026; C12Y 402/01107; C12N 1/20; C12Y 402/01017; C12P 7/16

(57) ABSTRACT

The use of microorganisms to make omega- and/or omega-1-functionalized products through an iterative carbon chain elongation pathway that we call a reverse beta oxidation pathway. The pathway uses omega-functionalized CoA thioesters as primers and acetyl-CoA as the extender unit in a non-decarboxylative Claisen condensation, and then uses beta oxidation or fatty acid synthesis enzymes to complete the cycle, via reductase, dehydratase and reductase reactions. Various termination enzymes that act on the functionalized beta-keto acyl-CoA intermediates of the pathway and produce omega or omega-1 functionalized products. The action of termination enzymes on such intermediates yield a large variety of products.

### PRIOR RELATED APPLICATIONS

This application claims is a Continuation-in-Part and claims priority and benefits under 35 U.S.C. § 119, 120 or 371 to U.S. Ser. No. 15/784,691, filed Oct. 16, 2017 (pending), titled SYNTHESIS OF OMEGA FUNCTIONALIZED PRODUCTS, which claims priority to:

U.S. Ser. No. 62/148,248, filed Apr. 16, 2015, and a continuation of PCT/US16/27903 filed Apr. 15, 2016, SYNTHESIS OF OMEGA FUNCTIONALIZED METHYLKETONES, 2-ALCOHOLS, 2-AMINES, AND DERIVATIVES THEREOF;

U.S. Ser. No. 62/154,397, filed Apr. 29, 2015, and a Continuation of PCT/US16/29592, filed Apr. 27, 2016, SYNTHESIS OF OMEGA-1 FUNCTIONALIZED PRODUCTS AND DERIVATIVES THEREOF; and

62/154,010, filed Apr. 28, 2015, and is a Continuation of PCT/US16/29583, filed Apr. 27, 2016, SYNTHESIS OF OMEGA-PHENYL PRODUCTS AND DERIVATIVES THEREOF;

### FEDERALLY SPONSORED RESEARCH STATEMENT

This invention was made with government support under Grant Nos. EEC-0813570, CBET-1134541, and CBET-1067565 awarded by the National Science Foundation. The government has certain rights in the invention.

each incorporated by reference herein in its entirety for all purposes.

### FIELD OF THE DISCLOSURE

The disclosure generally relates to the use of microorganisms to make omega- or omega-1-functionalized chemicals and fuels.

### BACKGROUND OF THE DISCLOSURE

Reactions that catalyze the iterative formation of carbon-carbon bonds are instrumental for many metabolic pathways, such as the biosynthesis of fatty acids, polyketides, and many other molecules with applications ranging from biofuels and green chemicals to therapeutic agents. These pathways typically start with small precursor metabolites that serve as building blocks that are subsequently condensed and modified in an iterative fashion until the desired chain length and functionality are achieved.

Most iterative carbon-carbon bond forming reactions in natural biological systems take place through a Claisen condensation mechanism in which the nucleophilic α-anion of an acyl-thioester, serving as the extender unit, attacks the electrophilic carbonyl carbon of another acyl-thioester, serving as the primer. Depending on how the nucleophilic α-anion is generated, the Claisen condensation reaction can be classified as decarboxylative or non-decarboxylative.

Many natural iterative carbon chain elongation pathways, like fatty acid and polyketide biosynthesis pathways, utilize decarboxylative Claisen condensation reactions with malonyl thioesters as extender units. Their potential products include fatty acids, alcohols, polyketides, esters, alkanes and alkenes with diverse chain lengths, structures and functionalities due to usage of functionalized primers, usage of α-functionalized malonyl thioesters as extender units and diverse pathways for termination of carbon chain elongation and subsequent product modification.

However, despite the structural and functional diversity of these products, the use of malonyl thioester as C2 extender unit requires the ATP-dependent activation of acetyl-CoA to malonyl-CoA, which in turn limits the energy efficiency of these pathways. Furthermore, owing to the decarboxylation mechanism, the β-site of extender units of the decarboxylative Claisen condensation must be a carboxylate group, restricting the range of extender units and potentially limiting the diversity of products that can be generated through these carbon chain elongation pathways.

In order to overcome this limitation, we have recently implemented a novel approach by driving beta-oxidation in reverse to make fatty acids instead of degrading them (see US20130316413, WO2013036812, each incorporated by reference in its entirety for all purposes).

Unlike the fatty acid biosynthesis pathway, the reversal of the β-oxidation cycle operates with coenzyme-A (CoA) thioester intermediates and uses acetyl-CoA directly for acyl-chain elongation (rather than first requiring ATP-dependent activation to malonyl-CoA). In these pathways, thiolases catalyze the non-decarboxylative Claisen condensation in which acetyl-CoA, instead of malonyl thioesters, serves as the extender unit, and subsequent β-reduction reactions by hydroxyacyl-CoA dehydrogenases (HACDs), enoyl-CoA hydratases (ECHs) and enoyl-CoA reductases (ECRs) enable iteration, although enzymes of the fatty acid synthesis pathway can be also used to catalyze these β-reduction reactions. See e.g., WO2015112988, US20160340699. Compared to pathways beginning with a decarboxylative Claisen condensation, these reverse beta oxidation (R-BOX) pathways are more energy efficient due to less ATP consumption for the supply of extender unit acetyl-CoA than malonyl thioesters.

However, to date the thiolases have only utilized acetyl-CoA as the extender unit, thus limiting the functionality of synthesized products. A novel non-decarboxylative Claisen condensation reaction able to accept wider range of functionalized primers and proceed in an iterative or cyclic manner is required to diversify the product range of carbon-chain elongation.

Thus, what is needed in the art are variations on the reverse beta oxidation pathways that allow the production of a broader range of chemicals.

### SUMMARY OF THE DISCLOSURE

The disclosure generally relates to the use of microorganisms to make omega-functionalized and omega-1 functionalized chemicals and fuels by an iterative carbon chain elongation pathway that uses omega- or omega-1-functionalized CoA thioesters as primers and acetyl-CoA as the extender unit, in combination with various termination enzymes that act on the omega-functionalized intermediates of the pathway. The action of these termination enzymes on such intermediates yields a wide variety of functionalized products.

The engineered pathway consists of five core enzymatic steps that generate omega-functionalized or omega-1 functionalized R-BOX intermediates of different carbon chain lengths. These will be abbreviated as “ω” and “ω-1” herein, together as “ω/ω-1” or just “omega functionalized.”

In the first step, ω/ω-1-functionalized CoA thioesters to be used as primers are generated, mainly by activation of their acid form, which can be either supplemented in the media or derived from carbon sources. Alternatively, these primers can be derived from carbon sources without this step.

Second, thiolase catalyzed non-decarboxylative Claisen condensation between ω/ω-1-functionalized primer and acetyl-CoA yields an ω/ω-1-functionalized β-keto acyl-CoA.

Further carbon chain elongation is achieved by subsequent dehydrogenation catalyzed by HACDs, dehydration catalyzed by ECHs and reduction catalyzed by ECRs and iterations of these reactions, which taken together generate ω/ω-1-functionalized intermediates of different carbon chain lengths.

These ω/ω-1-functionalized intermediates are then used as substrates for enzymes that convert them to different products. For example, coA can be removed from any of the pathway intermediates, and the resulting product collected as is or then can be further modified. Products can thus include:
omega-functionalized methyl ketones, 2-alcohols, 2-amines, and their derivatives, including lactams, lactones, α,ω-1-diamines, ω-1-amino-1-alcohols, ω-amino methyl ketones, ω-hydroxy methyl ketones, ω-amino-2-alcohols,
omega-1-functionalized carboxylic acids, alcohols, hydrocarbons, amines, and their beta-functionalized derivatives; and
omega-phenyl carboxylic acids, alcohols, amines, hydrocarbons, methyl ketones and their beta-functionalized derivatives

The process involves performing traditional fermentations using industrial organisms (such as E. coli, S. cerevisiae) that convert different feedstocks into longer-chain products. These organisms are considered workhorses of modern biotechnology. Media preparation, sterilization, inoculum preparation, and fermentation are the main steps of the process, once the requisite strains have been created.

As used herein, a “reverse beta oxidation pathway” is one that grows hydrocarbons by two carbon units and uses acetyl-CoA directly for acyl-chain elongation (rather than first requiring ATP-dependent activation to malonyl-CoA), and thus uses a non-decarboxylative Claisen condensation. Subsequent enzymes in the pathway can vary, and can include fatty acid synthesis enzymes as well as beta-oxidation enzymes. Repetitions of the pathway may be called cycles.

As used herein, a “primer” is a starting molecule for the iterative cycle to add two carbon donor units to a growing acyl-CoA thioester. The “initiating primer” can be any kind of omega-functionalized acyl-CoA. As the chain grows by adding donor units in each cycle, the primer will accordingly increase in size by 2C. In some cases, the bacteria can also be provided with larger initiating primers, e.g., C4 primers, etc. added to the media or obtained from other cell pathways. In this invention, non-traditional primers are used in which the terminal or penultimate carbon has been functionalized (i.e., omega methylated primers, omega-hydroxylated primers, omega-1-methylated primers, omega-1-hydroxylated primers, etc.). The ω/ω-1-functionalized initiating primers are either provided to the cell in the media, or made in the cell by the addition of appropriate enzymes, or combinations thereof (e.g., adding an omega-1-functionalized acid substrate that can be converted to its -CoA form in the cell).

As used herein, the “extender unit” is the donor of the 2 carbon units of each cycle of carbon elongation. In this disclosure, the extender unit is acetyl-CoA.

As used herein, the “omega” position is the last carbon in a straight chain, wherein the first position is determined by the -CoA activator. We do not change the nomenclature even after the -CoA is removed, where the nomenclature might otherwise change. An “omega-1-functionalized” group refers to a functional group (e.g. hydroxyl group) on the carbon one position over (closer to the -CoA) from the last carbon (e.g., the penultimate carbon in the straight chain or omega minus 1) in the straight chain, as referenced as the end opposite from where the -CoA is or was. As noted above, when we refer to omega and omega-1 together, we have used the abbreviation ω/ω-1 to include both positions.

As used herein “type II fatty acid synthesis enzymes” refer to those enzymes that function independently, e.g., are discrete, monofunctional enzymes, used in fatty acid synthesis. Type II enzymes are found in archaea and bacteria. Type I systems, in contrast, utilize a single large, multifunctional polypeptide. Type II enzymes can be used in the invention here for steps after the first condensation (e.g. beta-reduction steps).

Thiolases are ubiquitous enzymes that have key roles in many vital biochemical pathways, including the beta-oxidation pathway of fatty acid degradation and various biosynthetic pathways. Members of the thiolase family can be divided into two broad categories: degradative thiolases (EC 2.3.1.16), and biosynthetic thiolases (EC 2.3.1.9). The forward and reverse reactions are shown below:

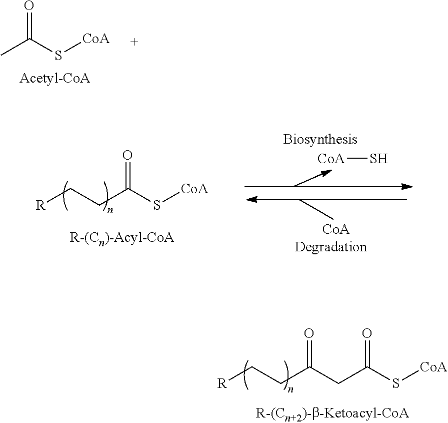

These two different types of thiolase are found both in eukaryotes and in prokaryotes:acetoacetyl-CoA thiolase (EC:2.3.1.9) and 3-ketoacyl-CoA thiolase (EC:2.3.1.16). 3-ketoacyl-CoA thiolase (also called thiolase I) has a broad chain-length specificity for its substrates and is involved in degradative pathways such as fatty acid beta-oxidation. Acetoacetyl-CoA thiolase (also called thiolase II) is specific for the thiolysis of acetoacetyl-CoA and involved in biosynthetic pathways such as poly beta-hydroxybutyric acid synthesis or steroid biogenesis.

Furthermore, the degradative thiolases can be made to run in the forward direction by building up the level of left hand side reactants (primer and extender unit), thus driving the equilibrium in the forward direction and/or by overexpressing same or by expressing a mutant of same.

As used herein, native or engineered “thiolases” able to use functionalized primers and extender units is an enzyme that catalyzes the condensation of ω/ω-1-functionalized acyl-CoA thioester with acetyl-CoA as the 2-carbon donor for chain elongation to produce an omega-functionalized β-keto acyl-CoA in a non-decarboxylative condensation reaction (R represents the functional group, here at omega-1, but it could also be in the omega position):

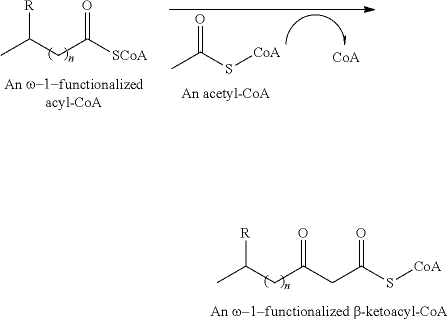

As used herein a “hydroxyacyl-CoA dehydrogenase” or “HACD”, is an enzyme that catalyzes the reduction of an ω/ω-1-functionalized β-keto acyl-CoA to a β-hydroxyacyl-CoA:

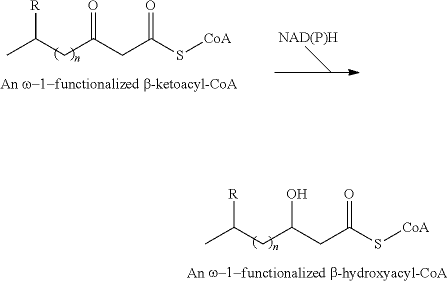

As used herein, “enoyl-CoA hydratase or “ECH” is an enzyme that catalyzes the dehydration of an ω/ω-1-functionalized β-hydroxyacyl-CoA to an omega-functionalized enoyl-CoA:

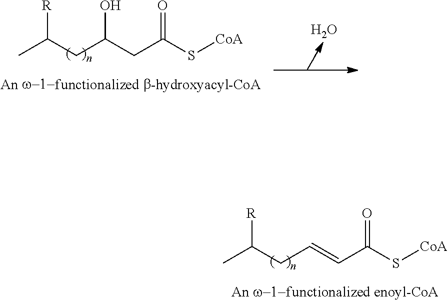

As used herein, an “enoyl-CoA reductase” or “ECR” is an enzyme that catalyzes the reduction of an ω/ω-1-functionalized trans-enoyl-CoA to an omega-functionalized acyl-CoA:

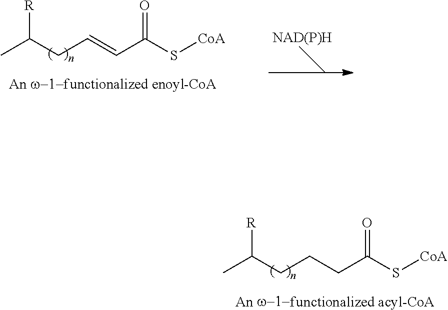

As used herein “termination pathway” refers to one or more enzymes (or genes encoding same) that will pull reaction CoA thioester intermediates out of the iterative cycle and produce the desired end product.

By “primary termination pathway” what is meant is a CoA thioester intermediate from the iterative cycle is pulled out of the iterative cycle by one (which can have more than one activity) or more termination enzymes and results in i) carboxylic acids, ii) primary alcohols, iii) hydrocarbons, iv) primary amines, or v) derivatives thereof from CoA thioesters intermediates.

By “secondary termination pathway” what is meant is that the intermediate pulled out of the iterative cycle by a primary termination pathway enzyme is further modified by one or more enzymes.

Many microbes do not make significant amounts of free fatty acids, but can be made to do so by adding a gene coding for an acyl-ACP thioesterase (called a “TE” gene herein), which are promiscuous enzymes that also work on -CoA activated intermediates, as well as ACP-carried intermediates in many cases. It is also known to change the chain length of the FFAs by changing the TE: 1) Class I acyl-ACP TEs act primarily on 14- and 16-carbon acyl-ACP substrates; 2) Class II acyl-ACP TEs have broad substrate specificities, with major activities toward 8- and 14-carbon acyl-ACP substrates; and, 3) Class III acyl-ACP TEs act predominantly on 8-carbon acyl-ACPs.

For example, most thioesterases exhibit the highest specificities in the C16-C18 range, including A. thaliana FatA (18:149), Madhuca longifolia FatB (16:0, 16:1, 18:0, 18:1), Coriandrum sativum FatA (18:149), A. thaliana FatB (16:0, 18:1, 18:0, 16:1), Helianthus annuus FatA (18:1, 16:1), and Brassica juncea FatB2 (16:0, 18:0), among numerous others. Medium-chain acyl-ACP thioesterases include Cuphea palustris FatB1 and C. hookeriana FatB2 (8:0, 10:0), C. palustris FatB2 (14:0, 16:0); and Umbellularia californica FatB (12:0, 12:1, 14:0, 14:1). Arecaceae (palm family) and Cuphea accumulate large quantities of fatty acids that are shorter (between 8 and 12 carbon atoms), and several enzymes are also available in bacteria. Thousands of such sequences are available.

As used herein, the expressions “microorganism,” “microbe,” “strain” and the like may be used interchangeably and all such designations include their progeny. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

As used herein, reference to a “cell” is generally understood to include a culture of such cells, as the work described herein is done in cultures having 10⁹⁻¹⁵ cells.

As used herein, “growing” cells is used in its art accepted manner, referring to exponential growth of a culture of cells, not the few cells that may not have completed their cell cycle at stationary phase or have not yet died in the death phase or after harvesting.

As used in the claims, “homolog” means an enzyme with at least 40% amino acid identity to one of the listed sequences and also having the same general catalytic activity, although of course Kₘ, K_{cat} and the like can vary. While higher identity (60%, 70%, 80%) and the like may be preferred, it is typical for bacterial sequences to diverge significantly (40-60%), yet still be identifiable as homologs, while mammalian species tend to diverge less (80-90%).

Reference to proteins herein can be understood to include reference to the gene encoding such protein. Thus, a claimed “permease” protein can include the related gene encoding that permease. However, it is preferred herein to refer to the protein by standard name per ecoliwiki or HUGO since both enzymatic and gene names have varied widely, especially in the prokaryotic arts.

Once an exemplary protein is obtained, many additional examples of proteins with similar activity can be identified by BLAST search. Further, every protein record is linked to a gene record, making it easy to design overexpression vectors. Many of the needed enzymes are already available in vectors, and can often be obtained from cell depositories or from the researchers who cloned them. But, if necessary, new clones can be prepared based on available sequence information using RT-PCR techniques. Thus, it should be easily possible to obtain all of the needed enzymes/genes for overexpression.

Another way of finding suitable enzymes/genes for use in the invention is to consider other enzymes with the same EC number, since these numbers are assigned based on the reactions performed by a given enzyme. An enzyme that thus be obtained, e.g., from AddGene or from the author of the work describing that enzyme, and tested for functionality as described herein. In addition, many sites provide lists of proteins that all catalyze the same reaction. If necessary, substrate specificity can be confirmed by testing a needed enzyme against a ω/ω-1-functionalized substrate in a bench top assay.

Understanding the inherent degeneracy of the genetic code allows one of ordinary skill in the art to design multiple nucleotides that encode the same amino acid sequence. NCBI™ provides codon usage databases for optimizing DNA sequences for protein expression in various species. Using such databases, a gene or cDNA may be “optimized” for expression in E. coli, yeast, algal or other species using the codon bias for the species in which the gene will be expressed.

Initial cloning experiments have proceeded in E. coli for convenience since most of the required genes are already available in plasmids suitable for bacterial expression, but the addition of genes to bacteria is of nearly universal applicability. Indeed, since recombinant methods were invented in the 70's and are now so commonplace, even school children perform genetic engineering experiments using bacteria. Such species include e.g., Bacillus, Streptomyces, Azotobacter, Trichoderma, Rhizobium, Pseudomonas, Micrococcus, Nitrobacter, Proteus, Lactobacillus, Pediococcus, Lactococcus, Salmonella, Streptococcus, Paracoccus, Methanosarcina, and Methylococcus, or any of the completely sequenced bacterial species. Indeed, hundreds of bacterial genomes have been completely sequenced, and this information greatly simplifies both the generation of vectors encoding the needed genes, as well as the planning of a recombinant engineering protocol. Such species are listed along with links at http://en.wikipedia.org/wiki/List_of_sequenced_bacterial_genomes.

Additionally, yeasts, such as Saccharomyces, are a common species used for microbial manufacturing, and many species can be successfully transformed. Indeed, yeast are already available that express recombinant thioesterases—one of the termination enzymes described herein—and the reverse beta oxidation pathway has already been achieved in yeast. Other species include but are not limited to Candida, Aspergillus, Arxula adeninivorans, Candida boidinii, Hansenula polymorpha (Pichia angusta), Kluyveromyces lactis, Pichia pastoris, and Yarrowia lipolytica, to name a few.

It is also possible to genetically modify many species of algae, including e.g., Spirulina, Apergillus, Chlamydomonas, Laminaria japonica, Undaria pinnatifida, Porphyra, Eucheuma, Kappaphycus, Gracilaria, Monostroma, Enteromorpha, Arthrospira, Chlorella, Dunaliella, Aphanizomenon, Isochrysis, Pavlova, Phaeodactylum, Ulkenia, Haematococcus, Chaetoceros, Nannochloropsis, Skeletonema, Thalassiosira, and Laminaria japonica, and the like. Indeed, the microalga Pavlova lutheri is already being used as a source of economically valuable docosahexaenoic (DHA) and eicosapentaenoic acids (EPA), and Crypthecodinium cohnii is the heterotrophic algal species that is currently used to produce the DHA used in many infant formulas.

Furthermore, a number of databases include vector information and/or a repository of vectors and can be used to choose vectors suitable for the chosen host species. See e.g., AddGene.org, which provides both a repository and a searchable database allowing vectors to be easily located and obtained from colleagues. See also Plasmid Information Database (PlasmID) and DNASU having over 191,000 plasmids. A collection of cloning vectors of E. coli is also kept at the National Institute of Genetics as a resource for the biological research community. Furthermore, vectors (including particular ORFS therein) are usually available from colleagues.

The enzymes can be added to the genome or via expression vectors, as desired. Preferably, multiple enzymes are expressed in one vector or multiple enzymes can be combined into one operon by adding the needed signals between coding regions. Further improvements can be had by overexpressing one or more, or even all of the enzymes, e.g., by adding extra copies to the cell via plasmid or other vector. Initial experiments may employ expression plasmids hosting 3 or more ORFs for convenience, but it may be preferred to insert operons or individual genes into the genome for long-term stability.

Still further improvements in yield can be had by reducing competing pathways, such as those pathways for making e.g., acetate, formate, ethanol, and lactate, and it is already well known in the art how to reduce or knockout these pathways. See e.g., the William Marsh Rice University patent portfolio by Ka-Yiu San and George Bennett (U.S. Pat. Nos. 7,569,380, 7,262,046, 8,962,272, 8,795,991) and patents by these inventors (U.S. Pat. Nos. 8,129,157 and 8,691,552) (each incorporated by reference herein in its entirety for all purposes). Many others have worked in this area as well.

In calculating “% identity” the unaligned terminal portions of the query sequence are not included in the calculation. The identity is calculated over the entire length of the reference sequence, thus short local alignments with a query sequence are not relevant (e.g., % identity=number of aligned residues in the query sequence/length of reference sequence). Alignments are performed using BLAST homology alignment as described by Tatusova TA & Madden TL (1999) FEMS Microbiol. Lett. 174:247-250, and available through the NCBI website. The default parameters were used, except the filters were turned OFF.

“Operably associated” or “operably linked”, as used herein, refer to functionally coupled nucleic acid or amino acid sequences.

“Recombinant” is relating to, derived from, or containing genetically engineered material. In other words, the genetics of an organism was intentionally manipulated by the hand-of-man in some way.

“Reduced activity” is defined herein to be at least a 75% reduction in protein activity, as compared with an appropriate control species (e.g., the wild type gene in the same host species). Preferably, at least 80, 85, 90, 95% reduction in activity is attained, and in the most preferred embodiment, the activity is eliminated (100%). Proteins can be inactivated with inhibitors, by mutation, or by suppression of expression or translation, by knock-out, by adding stop codons, by frame shift mutation, and the like. All reduced activity genes or proteins are signified herein by “−”.

By “null” or “knockout” what is meant is that the mutation produces undetectable active enzyme. A gene can be completely (100%) reduced by knockout or removal of part of all of the gene sequence. Use of a frame shift mutation, early stop codon, point mutations of critical residues, or deletions or insertions, and the like, can also completely inactivate (100%) gene product by completely preventing transcription and/or translation of active protein. All null mutants herein are signified by A.

“Overexpression” or “overexpressed” is defined herein to be at least 150% of protein activity as compared with an appropriate control species, or any detectable expression in a species that normally lacks that enzyme. Preferably, the activity is increased 100-500% or even ten-fold. Overexpression can be achieved by: mutating the protein to produce a more active form or a form that is resistant to inhibition, by removing inhibitors, or adding activators, and the like. Overexpression can also be achieved by removing repressors, adding multiple copies of the gene to the cell, or up-regulating the endogenous gene, and the like. All overexpressed genes or proteins are signified herein by “+”.

In certain species it is possible to genetically engineer the endogenous protein to be overexpressed by changing the regulatory sequences or removing repressors. However, overexpressing the gene by inclusion on selectable plasmids or other vectors that exist in hundreds of copies in the cell may be preferred due to its simplicity and ease of exerting externals controls, although permanent modifications to the genome may be preferred in the long term for stability reasons.

The term “endogenous” or “native” means that a gene originated from the species in question, without regard to subspecies or strain, although that gene may be naturally or intentionally mutated, or placed under the control of a promoter that results in overexpression or controlled expression of said gene. Thus, genes from Clostridia would not be endogenous to Escherichia, but a plasmid expressing a gene from E. coli or would be considered to be endogenous to any genus of Escherichia, even though it may now be overexpressed.

“Expression vectors” are used in accordance with the art-accepted definition of a plasmid, virus or other propagatable sequence designed for protein expression in cells. There are thousands of such vectors commercially available, and typically each has an origin of replication (ori); a multiple cloning site; a selectable marker; ribosome binding sites; a promoter and often enhancers; and the needed termination sequences. Most expression vectors are inducible, although constitutive expression vectors also exist.

As used herein, “inducible” means that gene expression can be controlled by the hand-of-man, by adding e.g., a ligand to induce expression from an inducible promoter. Exemplary inducible promoters include the lac operon, inducible by IPTG, the yeast AOX1 promoter inducible with methanol, the strong LAC4 promoter inducible with lactate, and the like. Low level of constitutive protein synthesis may occur even in expression vectors with tightly controlled promoters.

As used herein, an “integrated sequence” means the sequence has been integrated into the host genome, as opposed to being maintained on an expression vector. It will still be expressible, and preferably is inducible as well.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed, and excludes all additional elements.

The phrase “consisting essentially of” excludes additional material elements, but allows the inclusions of non-material elements that do not substantially change the nature of the invention, such as instructions for use, buffers, background mutations that do not affect the invention, and the like.

The following abbreviations are used herein:

| ABBREVIATION | TERM |
|---|---|
| ACP | Acyl carrier protein |
| ACR | Acyl-CoA reductase |
| ACT | Acyl-CoA thioesterase or Acyl-CoA transferase or |
| | phosphotransacylase + kinase |
| ADH | Alcohol dehydrogenase |
| CoA | Coenzyme A |
| DC | Decarboxylase |
| ECH | Enoyl-CoA hydratase |
| ECR | Enoyl-CoA reductase |
| FAS | Fatty acid biosynthesis |
| HACD | Hydroxyacyl-CoA dehydrogenases |
| R-Box or Box-R | Reverse Beta oxidation pathway |
| β-hydroxy | beta-hydroxy |
| ω | Omega, ultimate carbon at end opposite CoA |
| ω-1 | Omega minus 1, penultimate carbon |
| ω/ω-1 | Omega or omega-1 |

### BRIEF DESCRIPTION OF THE DRAWINGS

FIG. 1A-B. (81): Platform for the synthesis of ω/ω-1-functionalized products, where the ω/ω-1-functionalized primer is mainly activated from its acid form, which can be either supplemented in the media or derived from carbon sources, catalyzed by CoA-synthase, CoA transferase or phosphotransacylase+kinase. Primer can also be derived from carbon sources without this step. Condensation between ω/ω-1-functionalized primer and acetyl-CoA catalyzed by thiolase forms ω/ω-1-functionalized β-keto acyl-CoA. Further carbon chain elongation is achieved by subsequent reactions by dehydrogenase, dehydratase and reductase and iterations of the cycle.

Termination pathways are quite diverse, but exemplary termination pathways include CoA removal by thioesterase or CoA transferase and phosphotransacylase+kinase and decarboxylation by decarboxylase generate ω/ω-1-functionalized methyl ketone from ω/ω-1-functionalized β-keto acyl-CoA. Subsequent dehydrogenation by keto-dehydrogenase and amino group transfer by transaminase convert ω/ω-1-functionalized methyl ketone into ω/ω-1-functionalized 2-alcohol and 2-amine respectively.

R means functional group and n means length of primers, intermediates and products. Dashed line means multiple reaction steps or iteration. Here, we showed only the ω functional groups, but it is understood that the figure applies to both ω and ω-1 functionalized primers, intermediates and products.

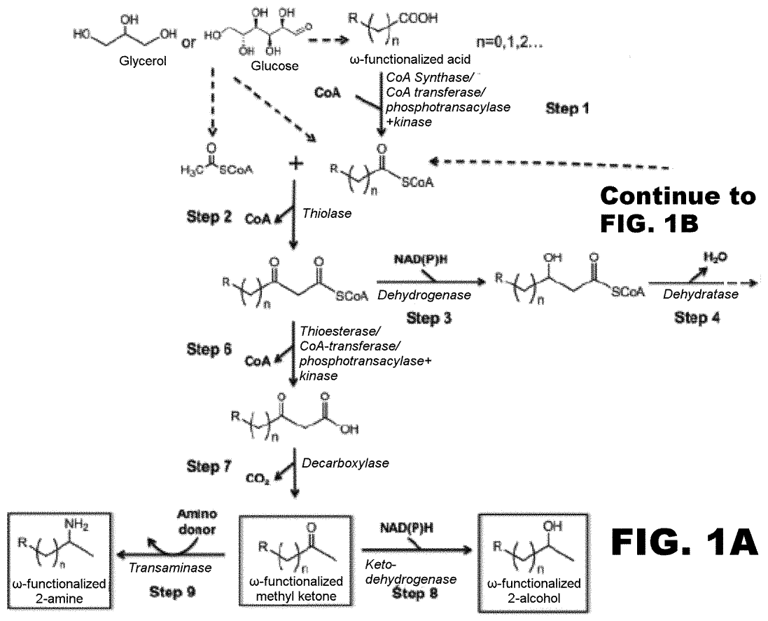

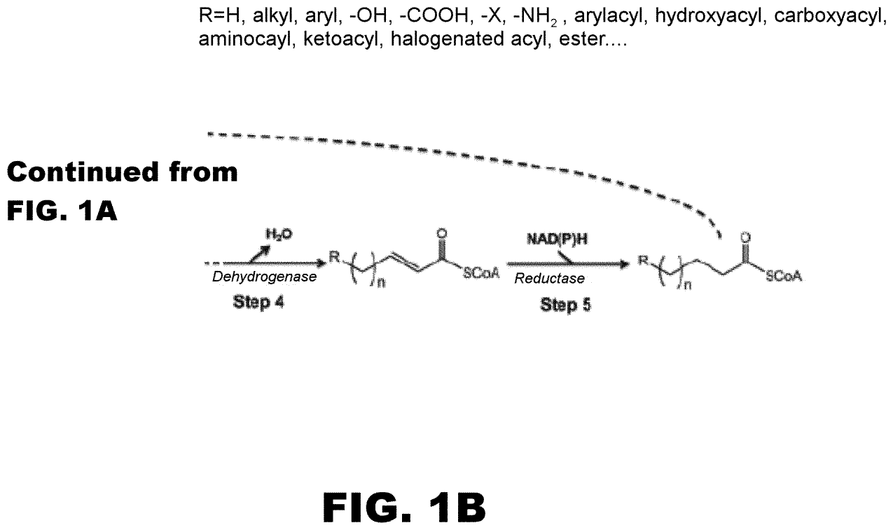

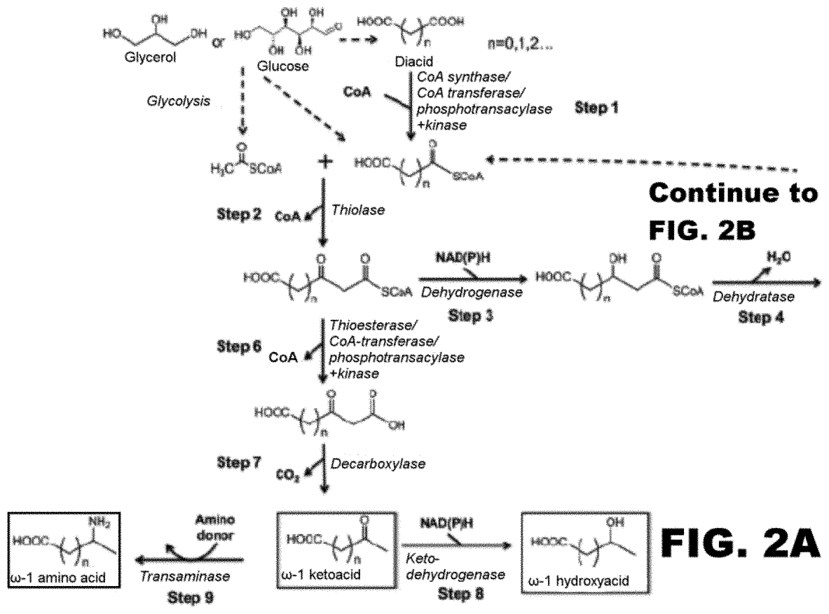

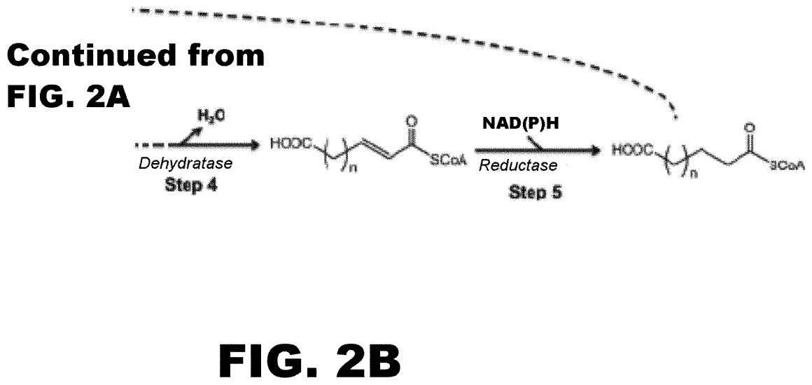

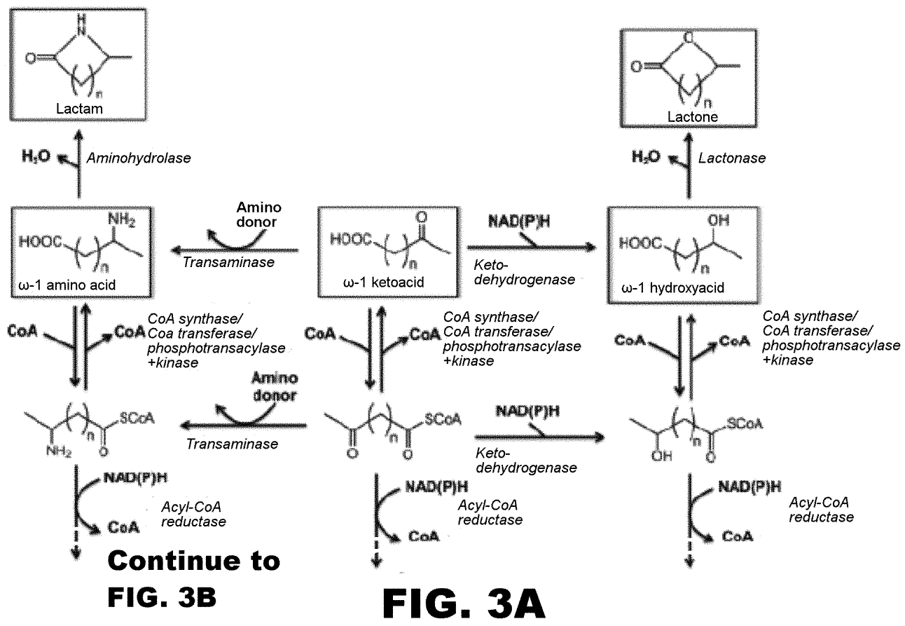

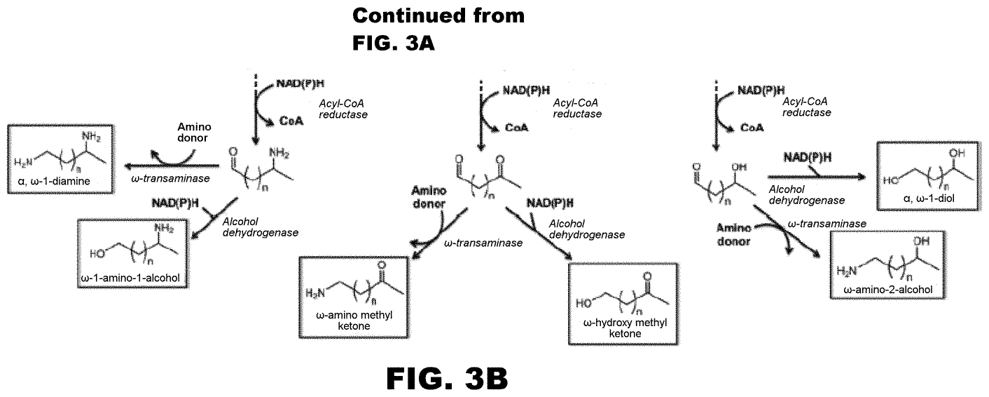

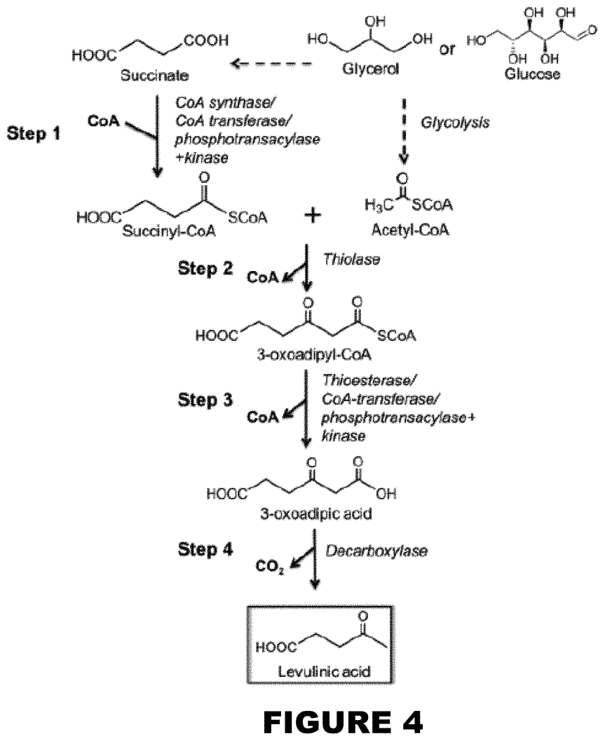

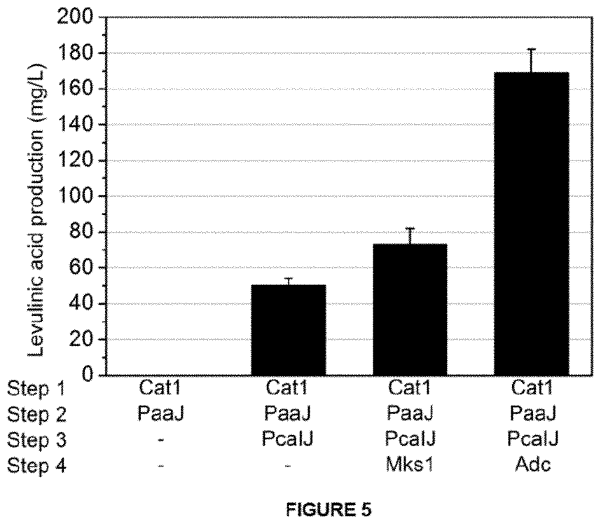

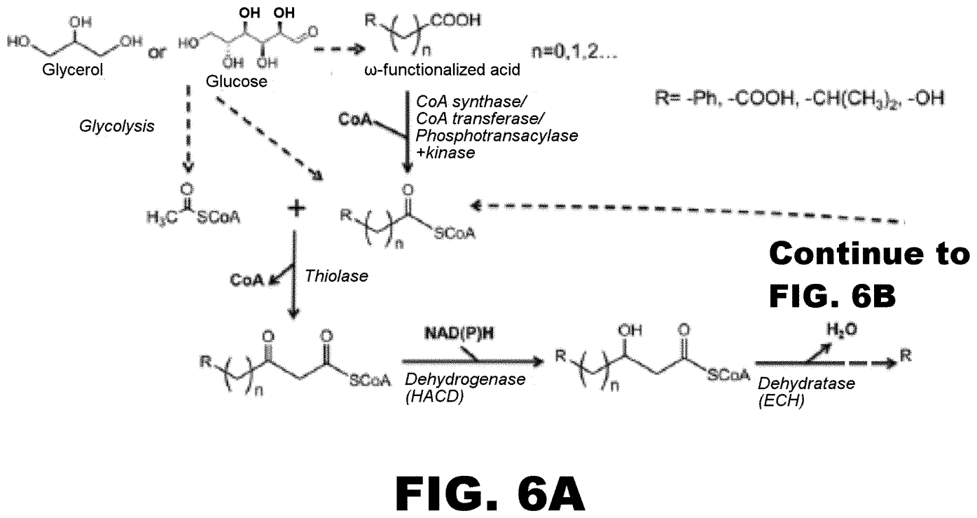

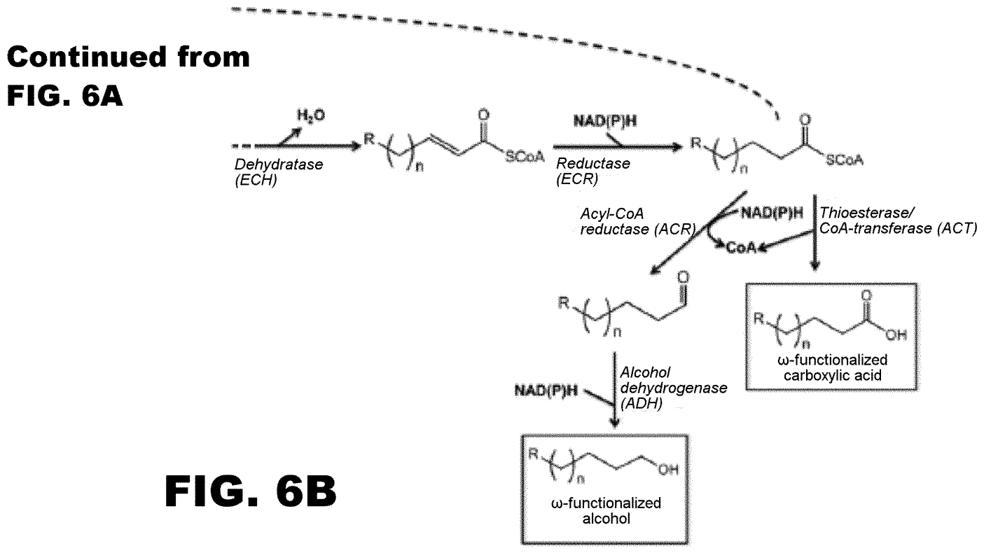

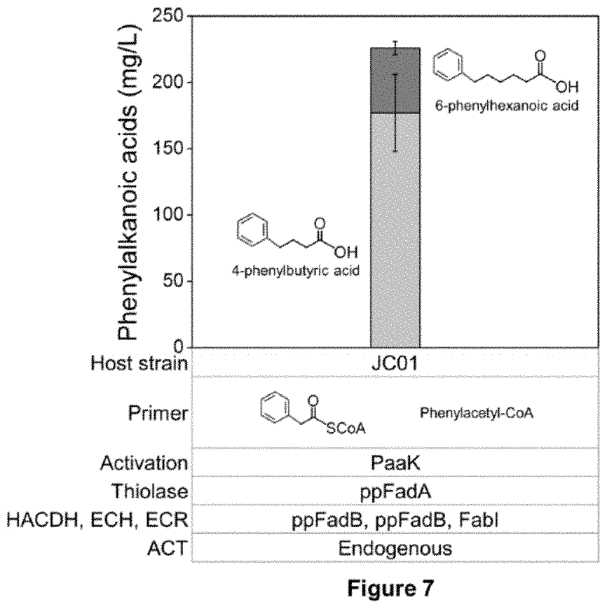

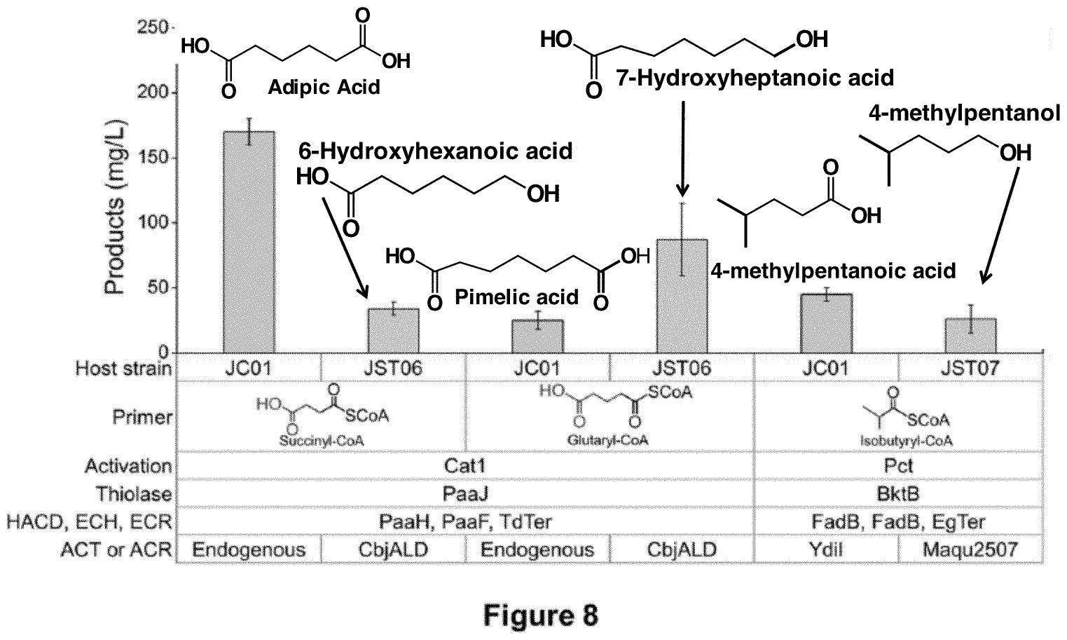

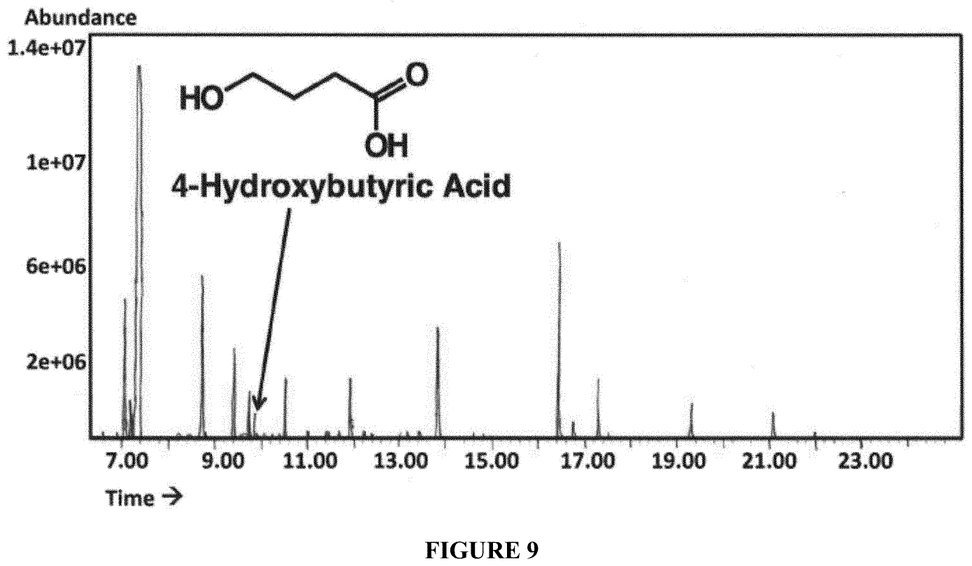

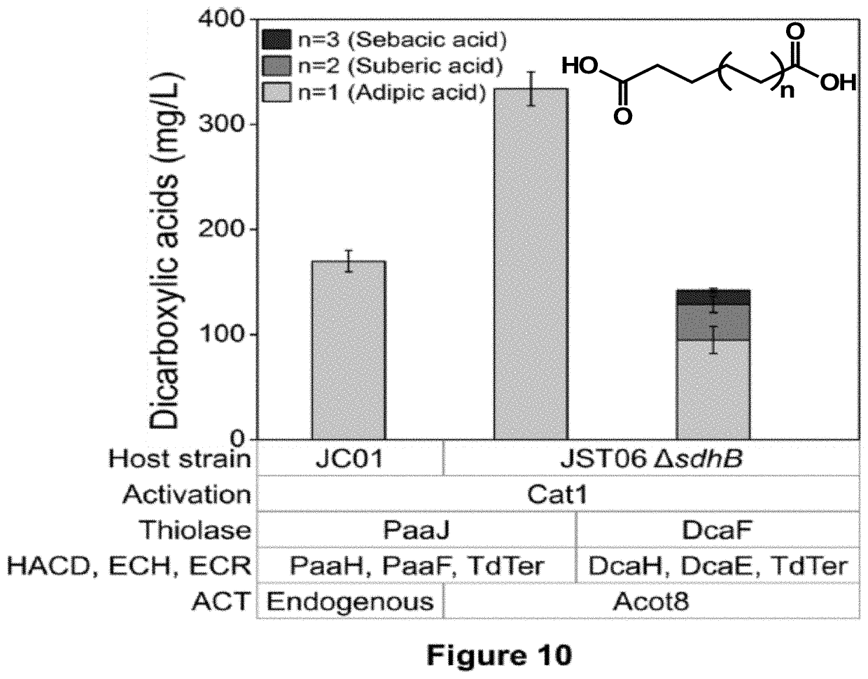

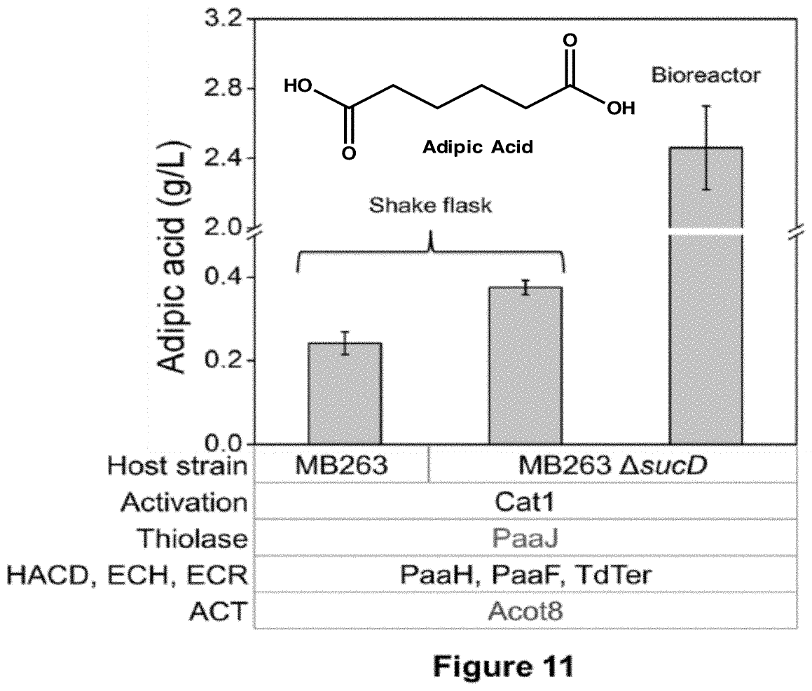

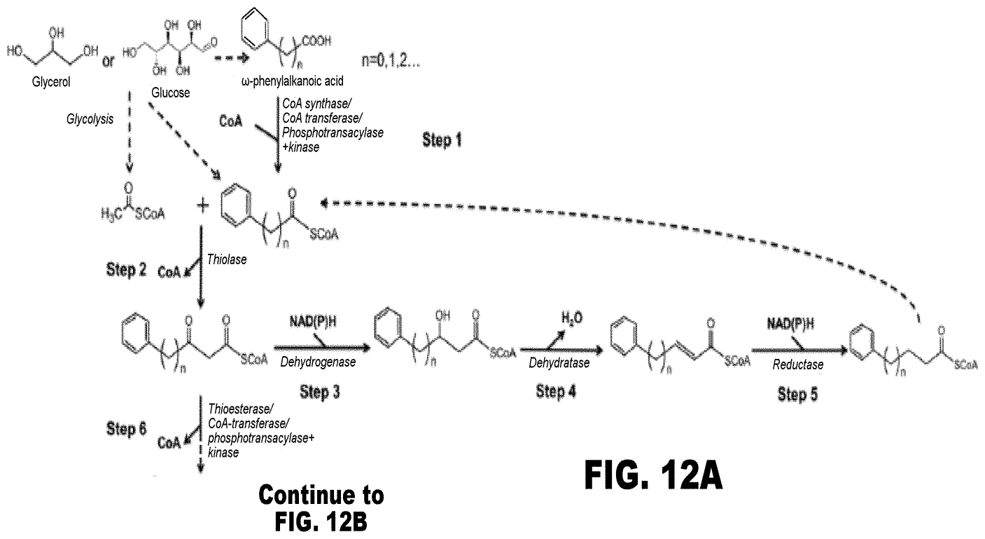

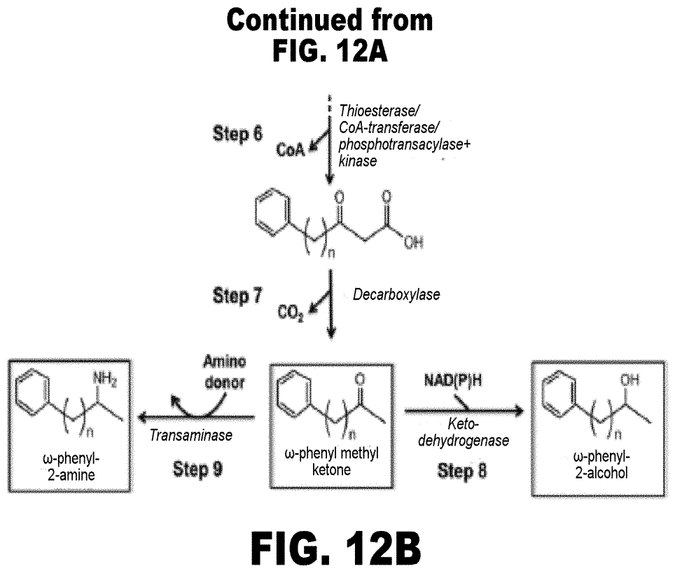

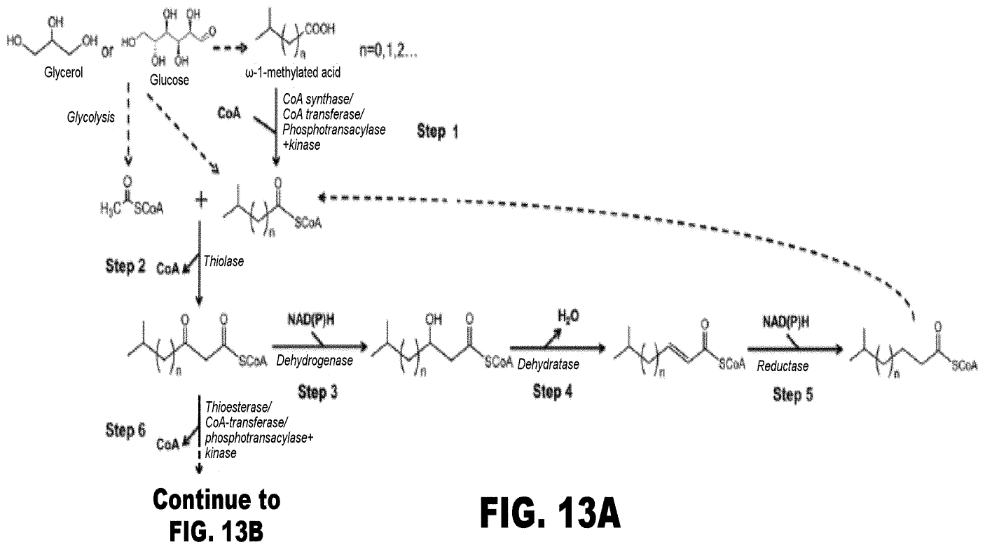

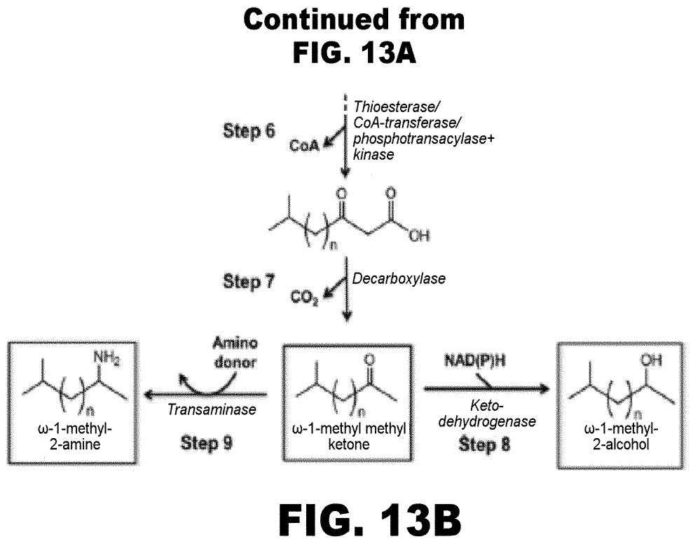

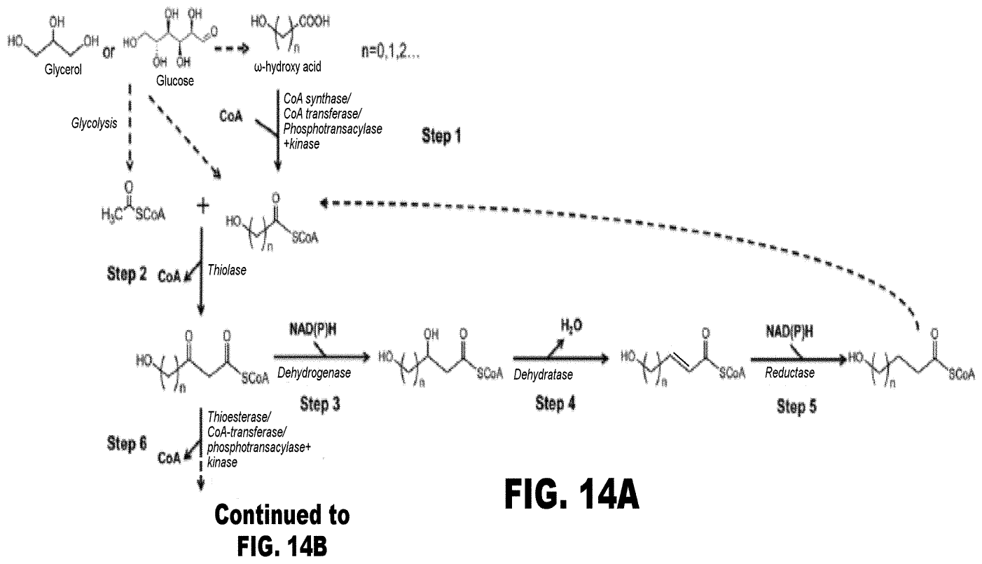

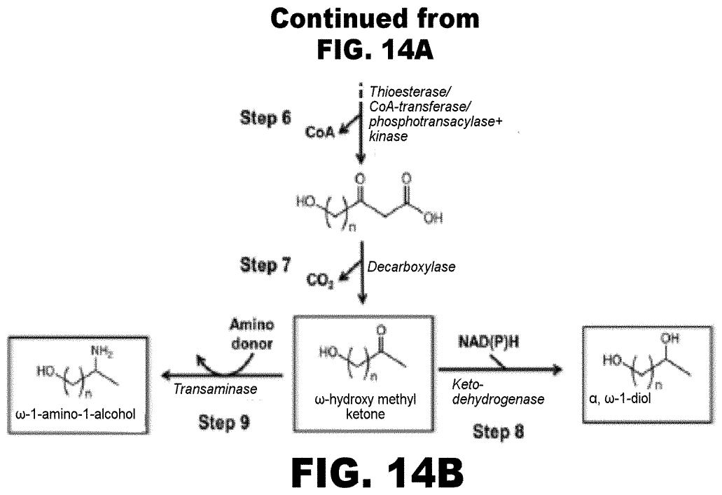

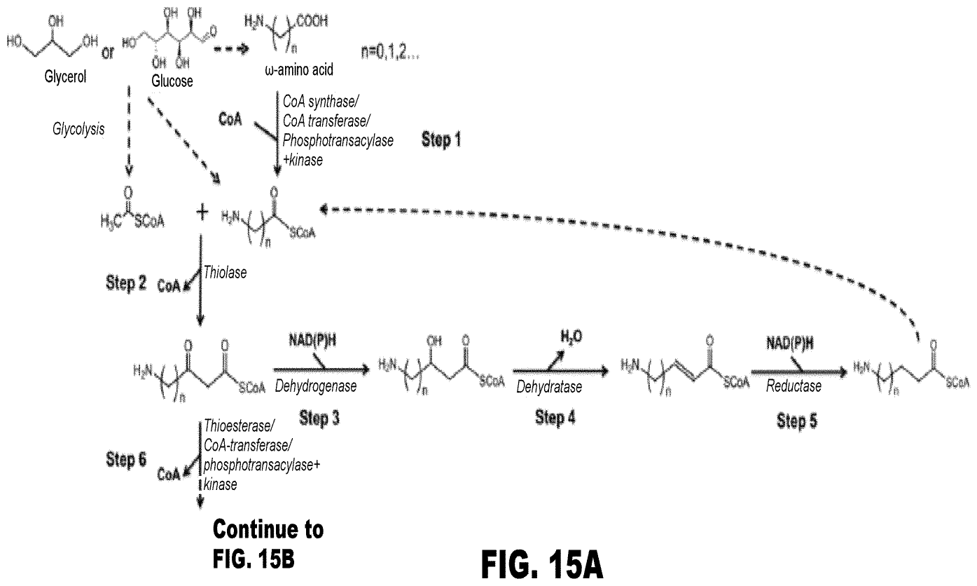

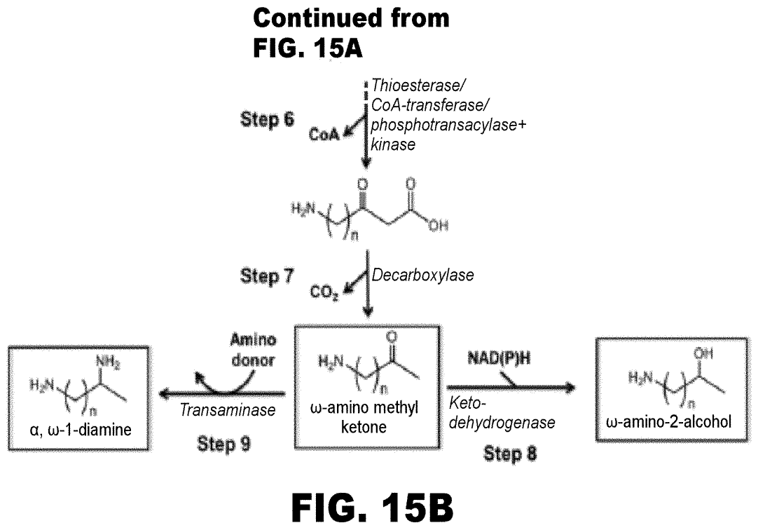

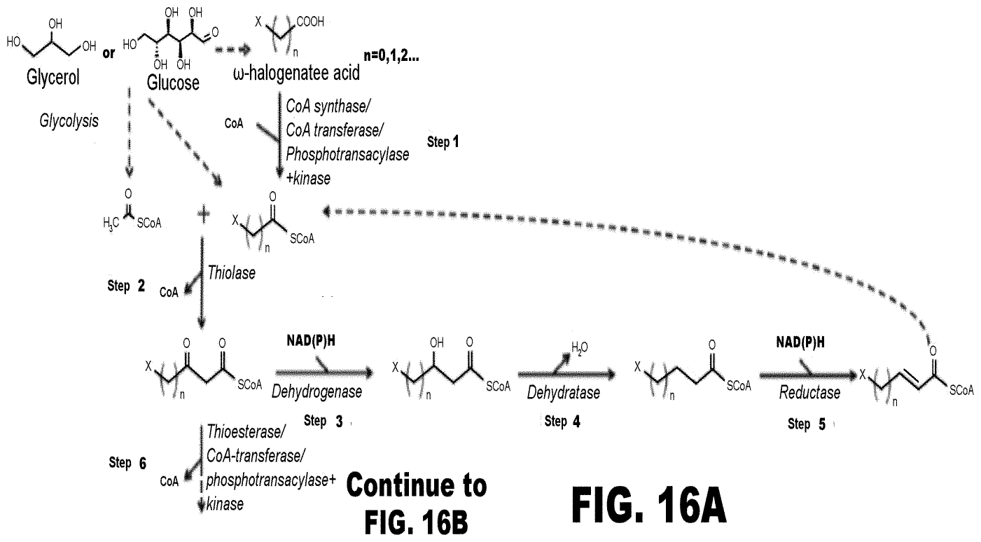

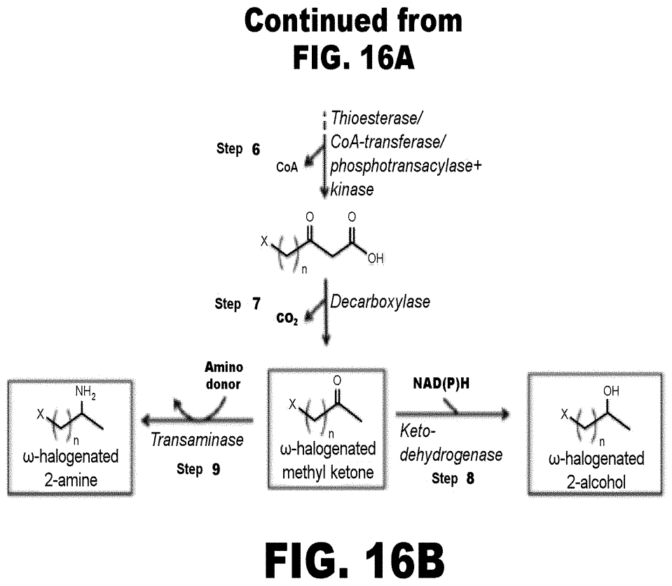

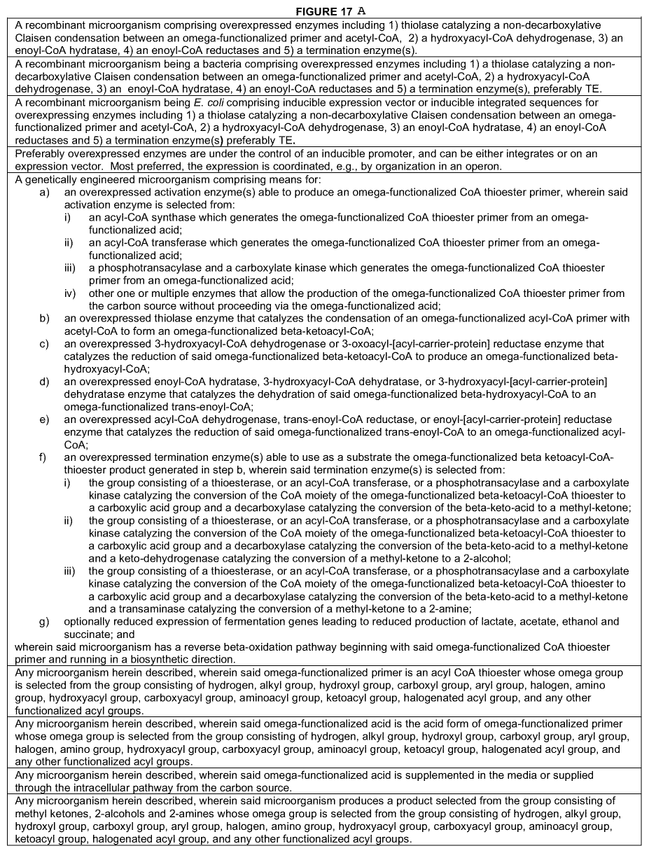

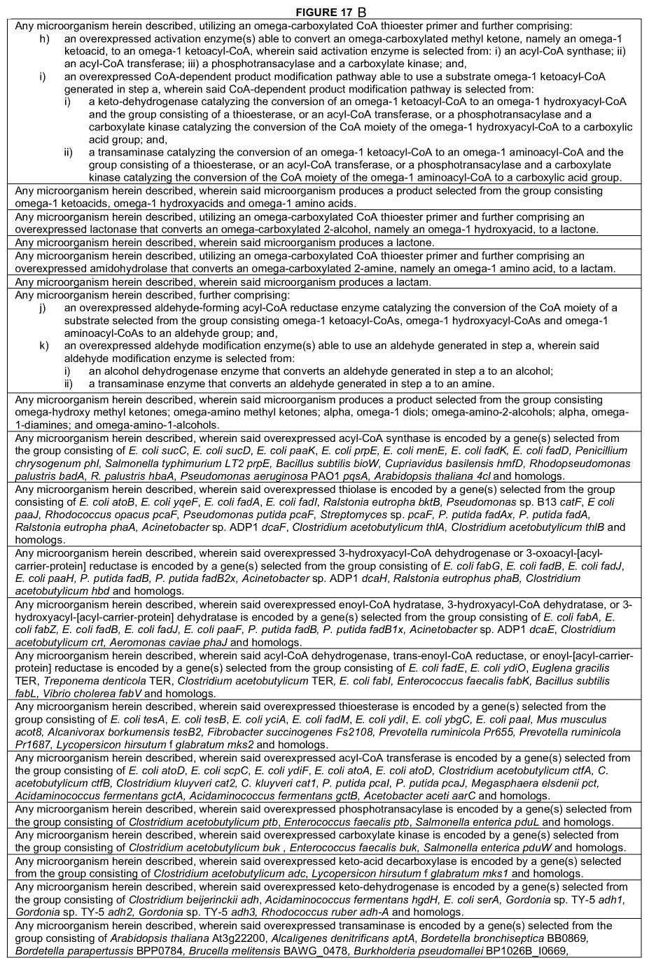

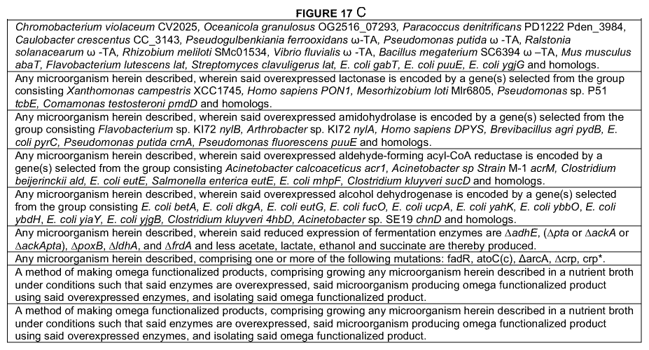

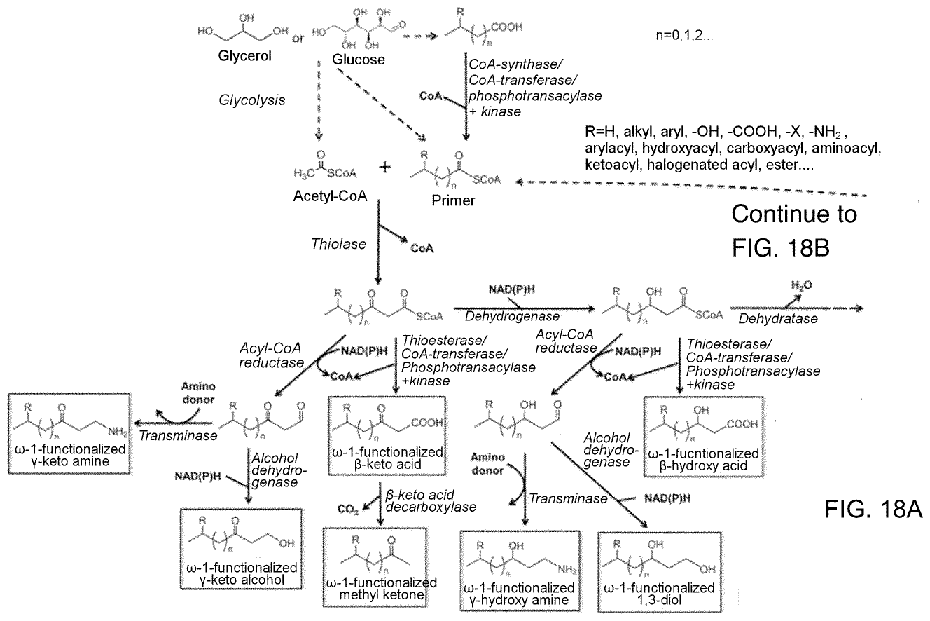

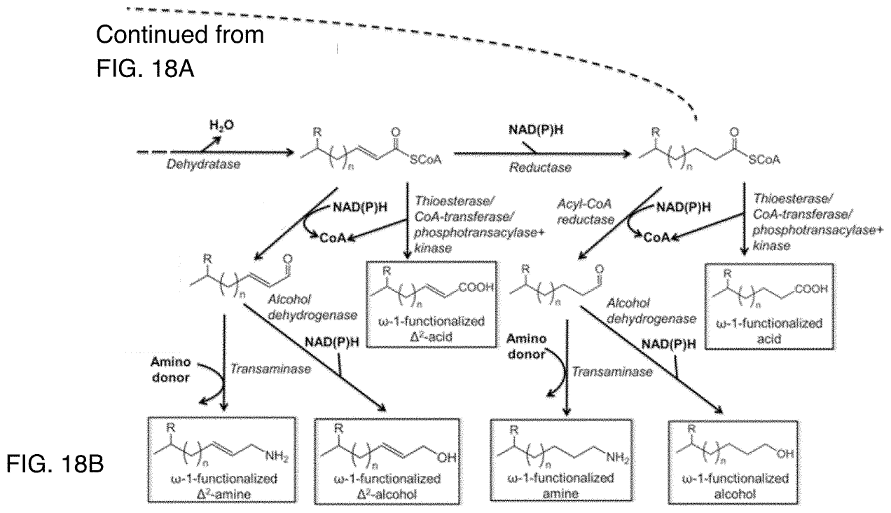

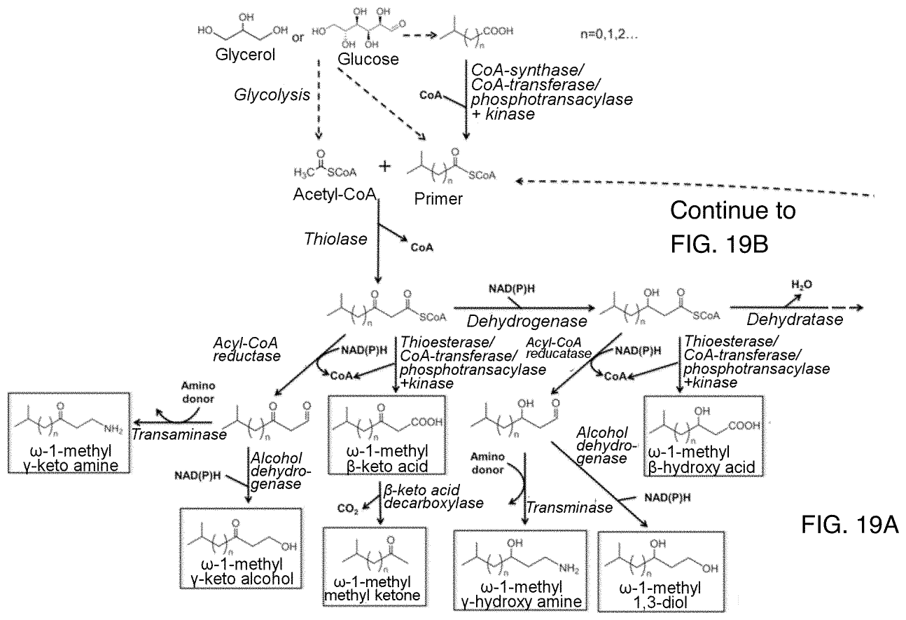

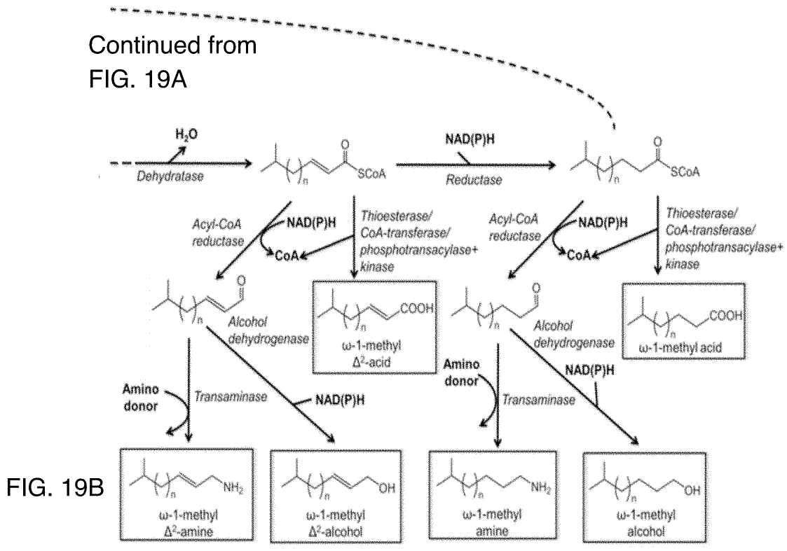

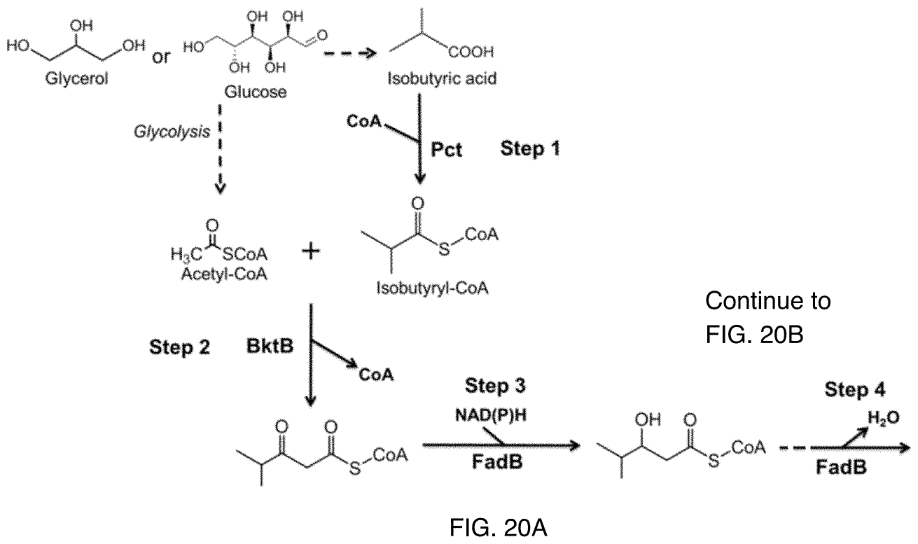

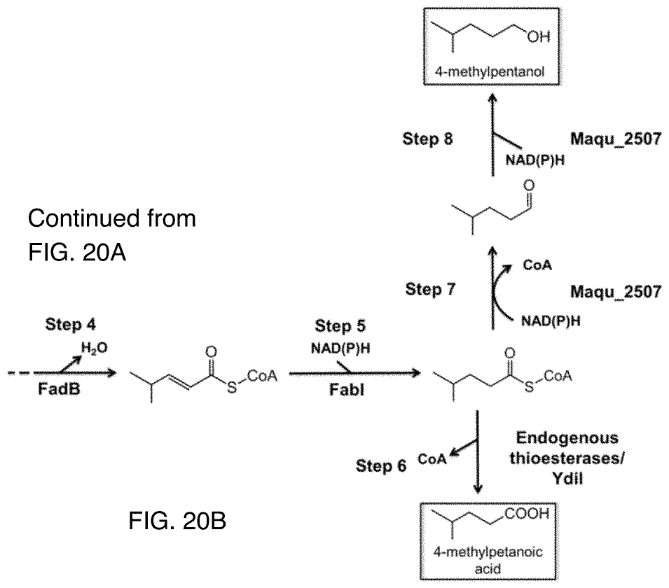

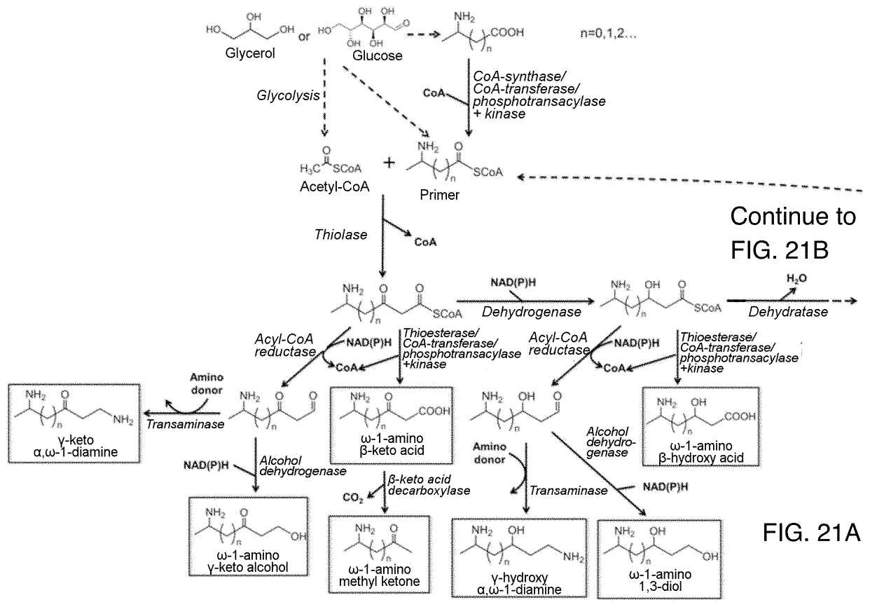

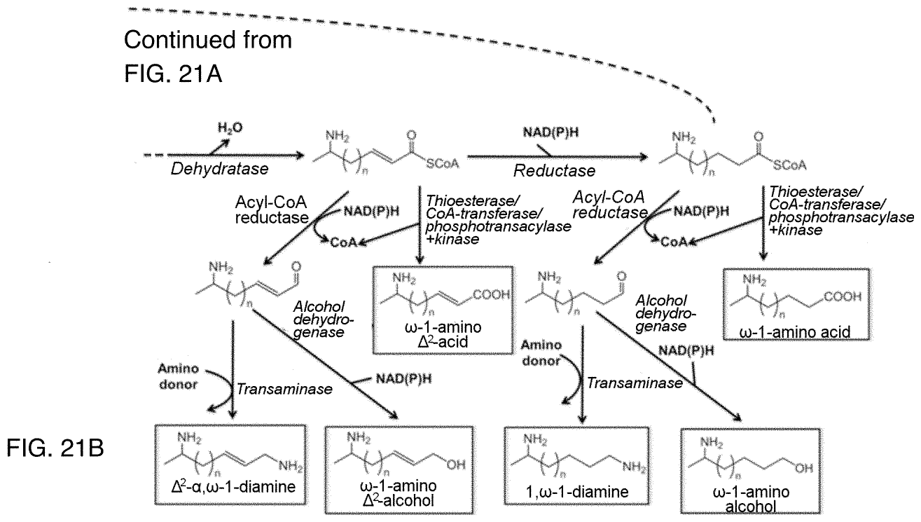

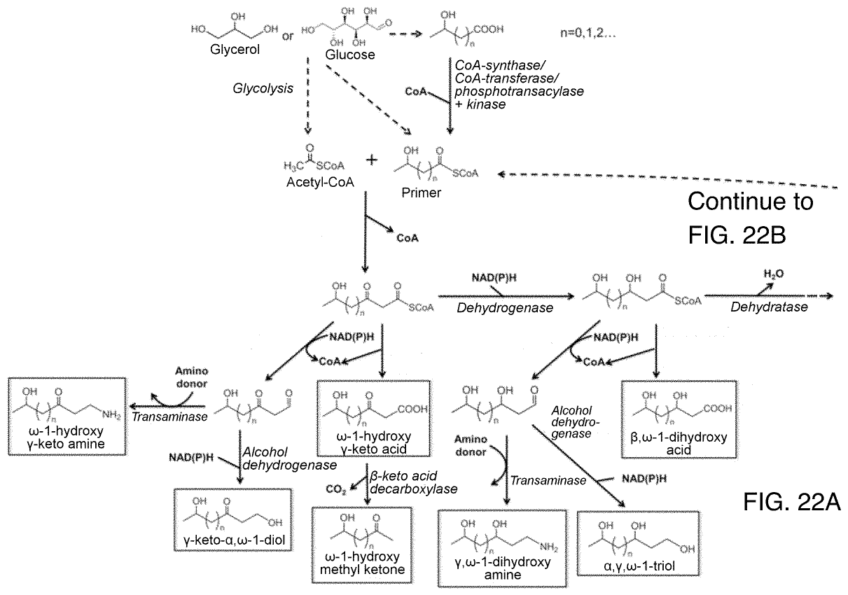

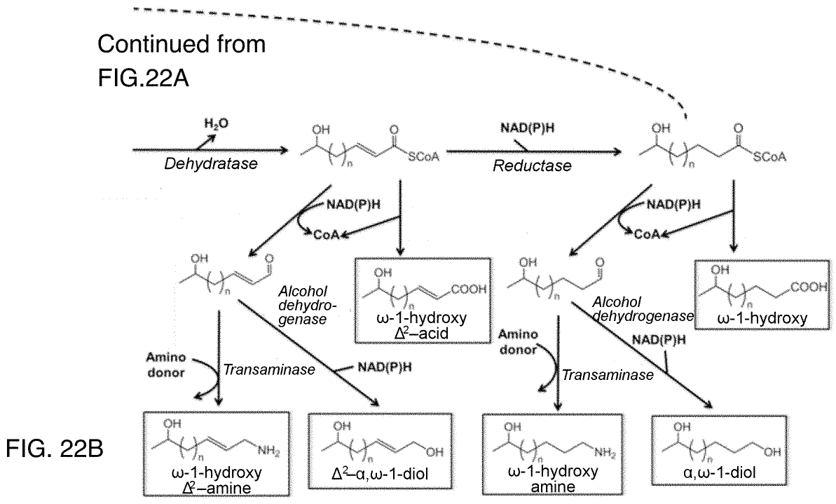

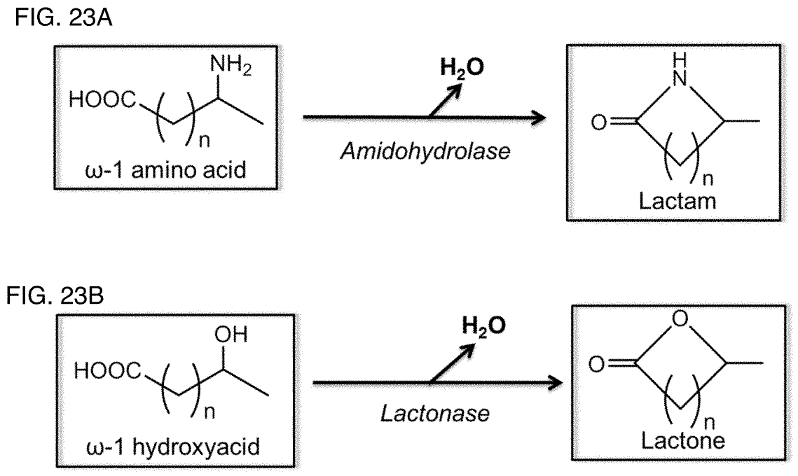

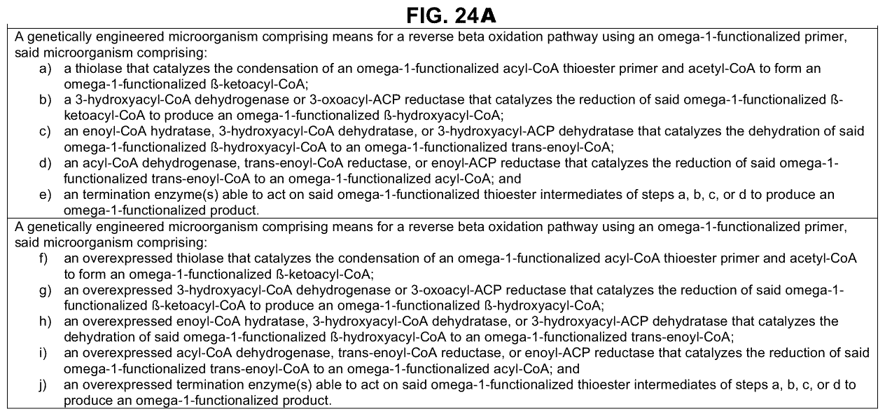

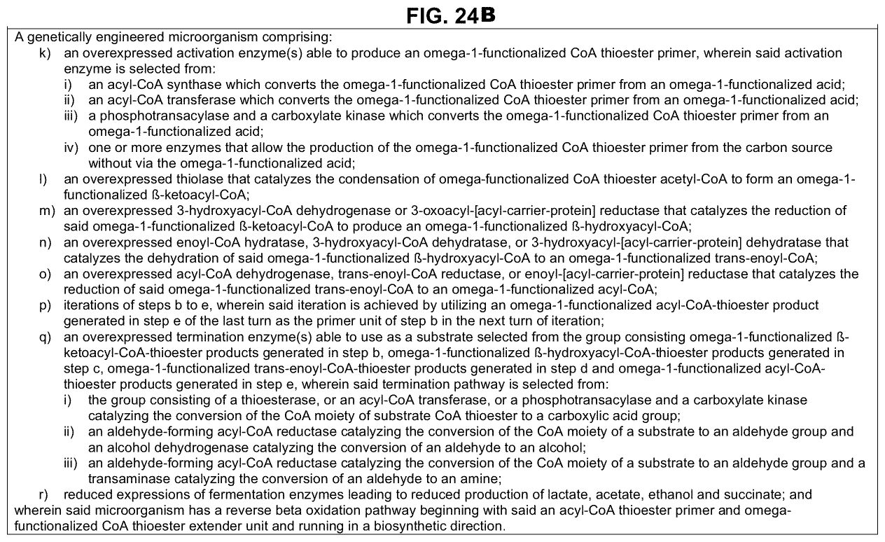

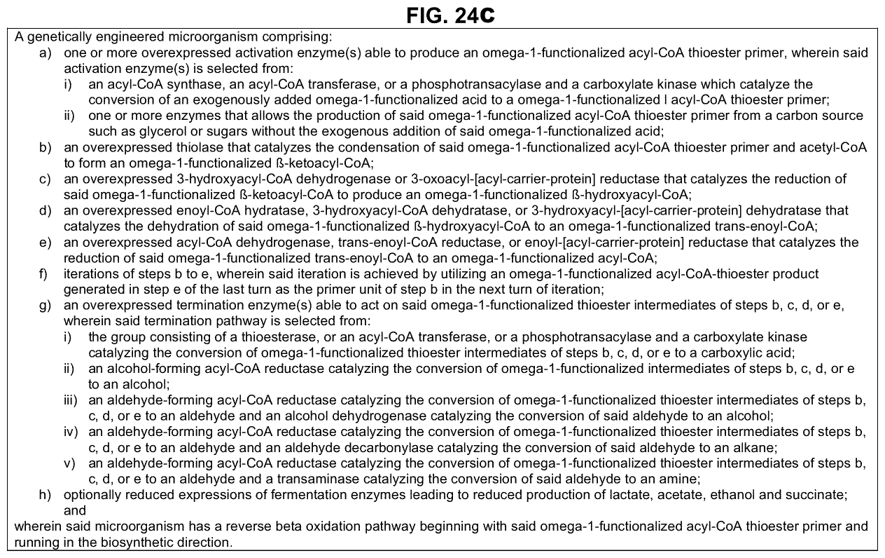

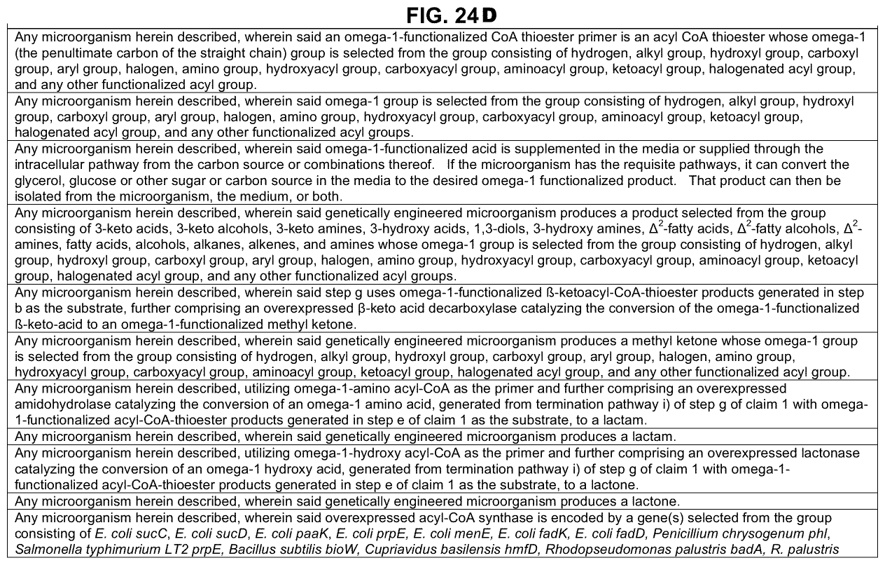

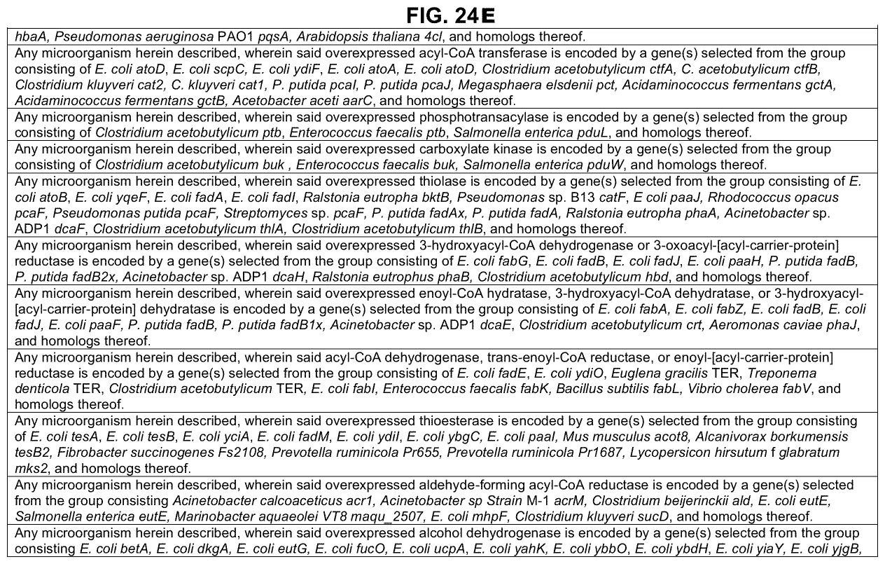

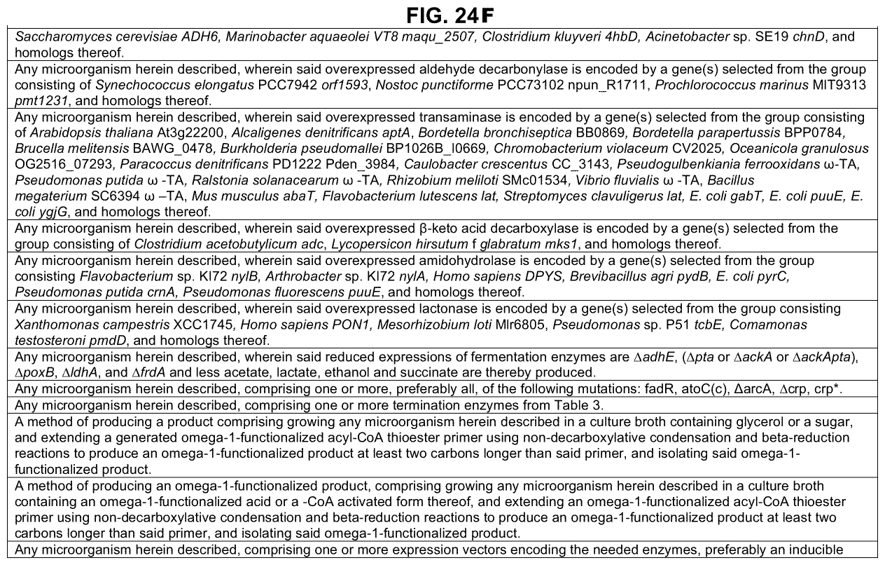

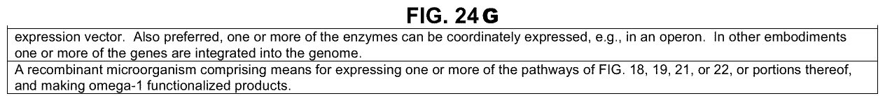

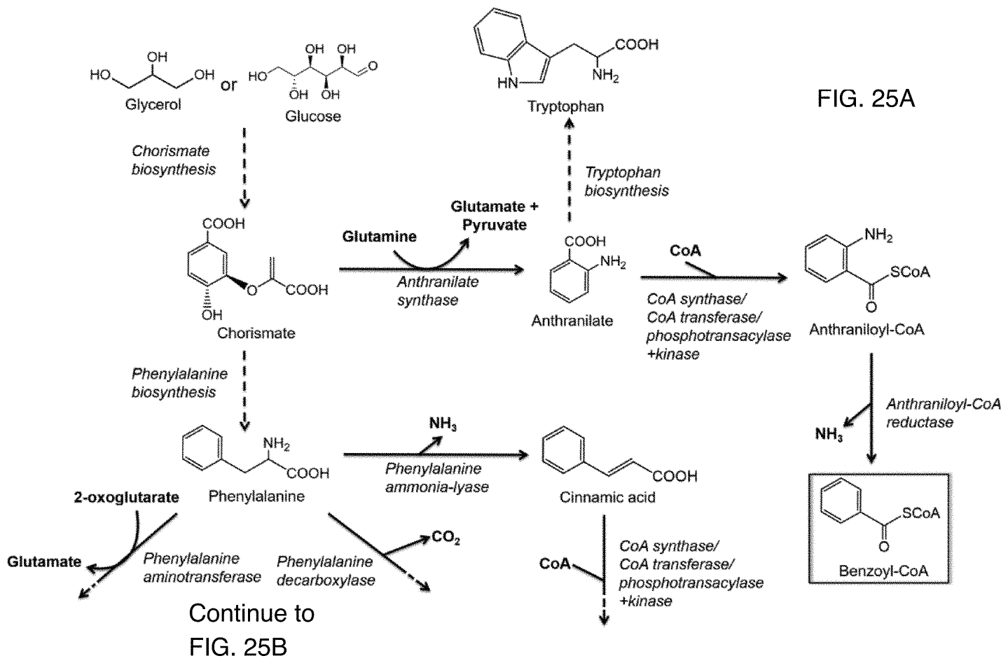

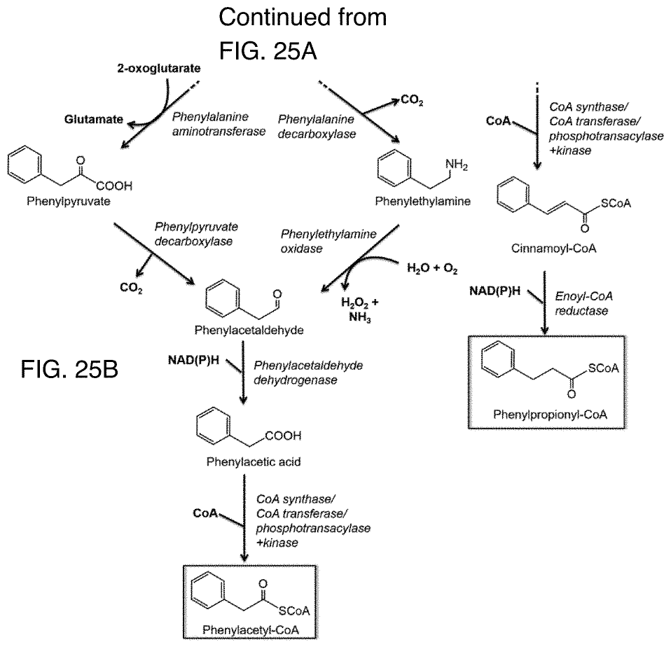

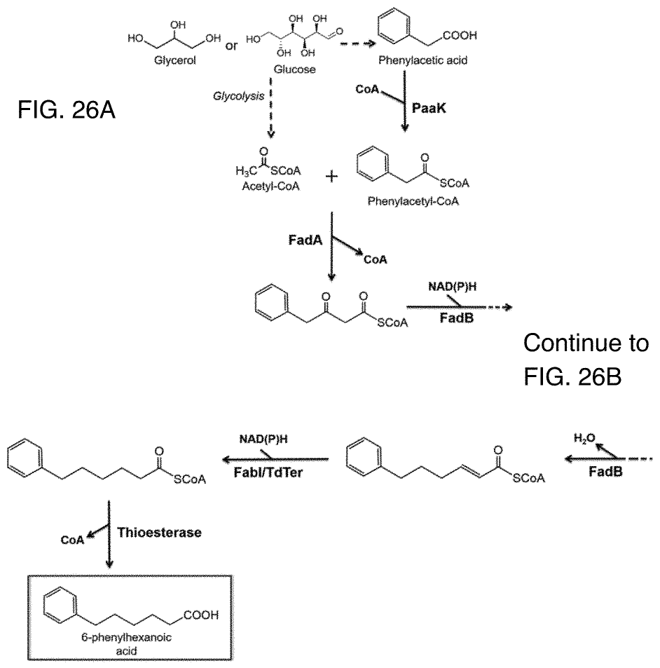

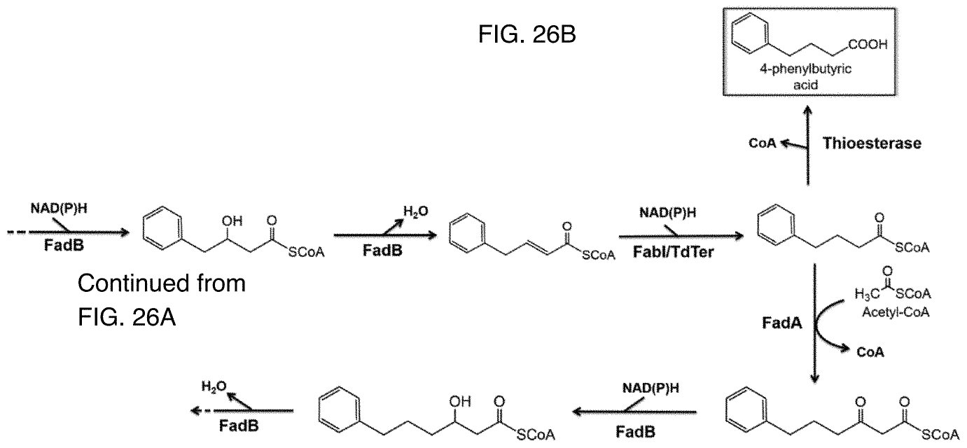

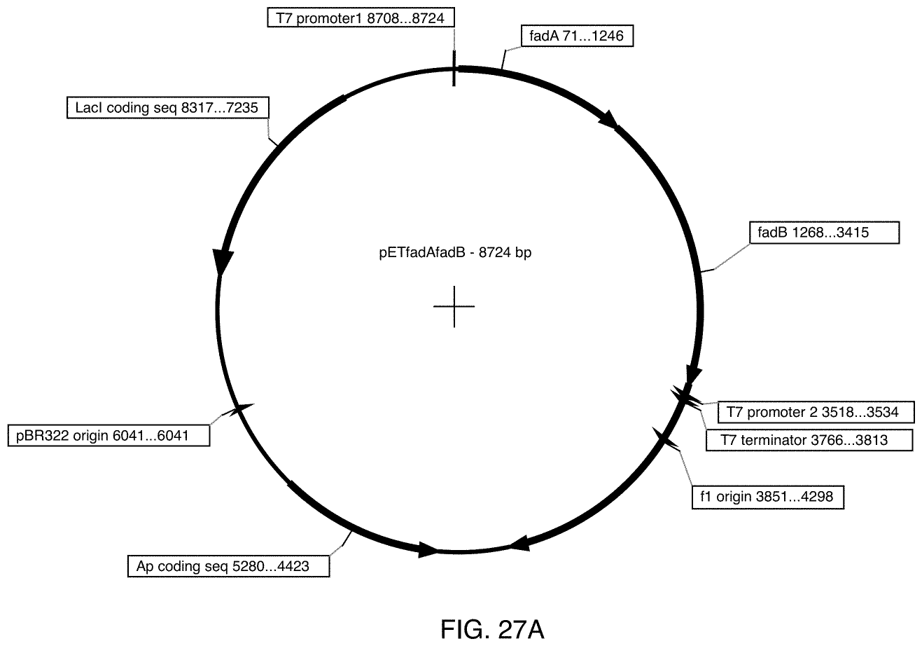

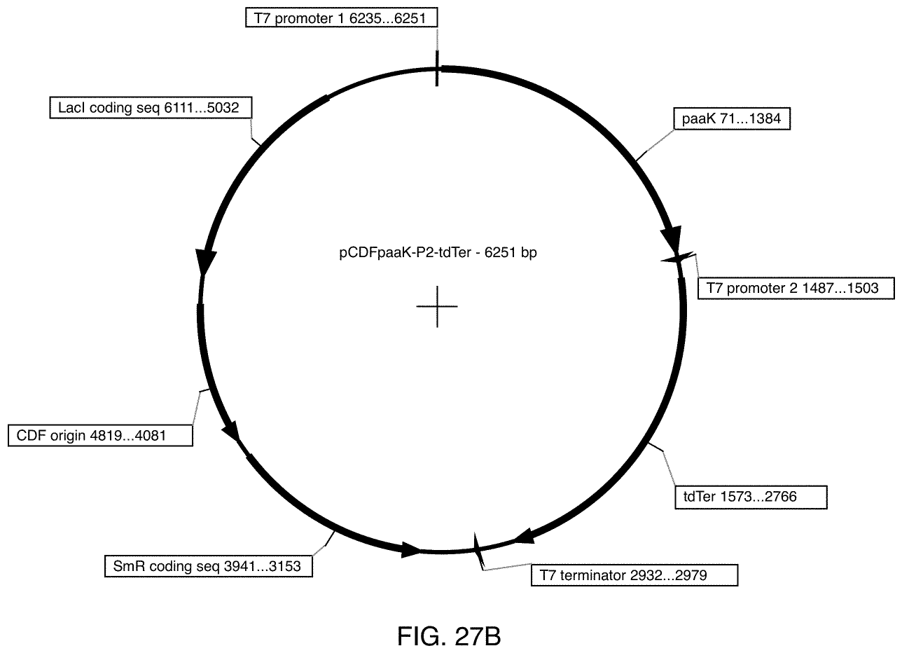

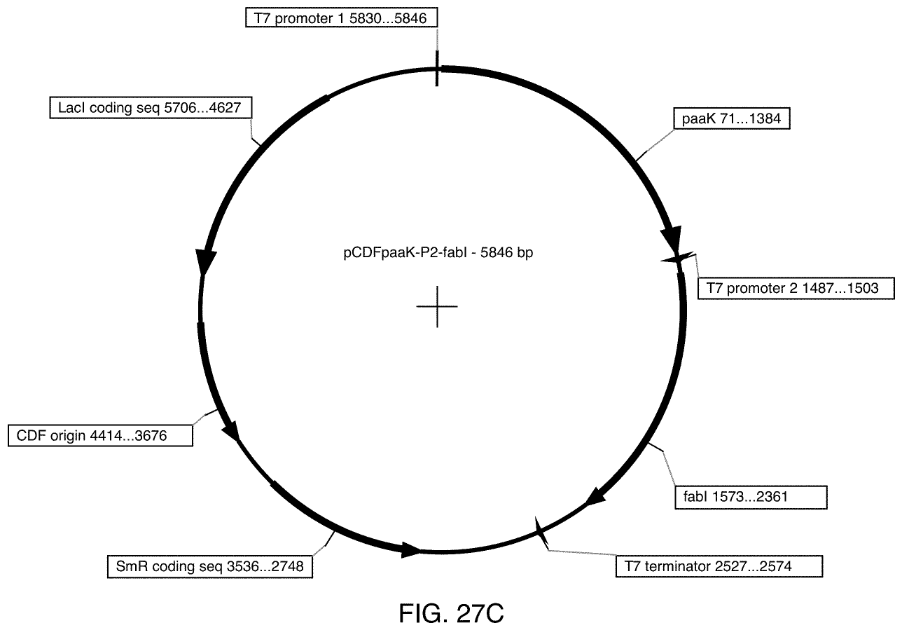

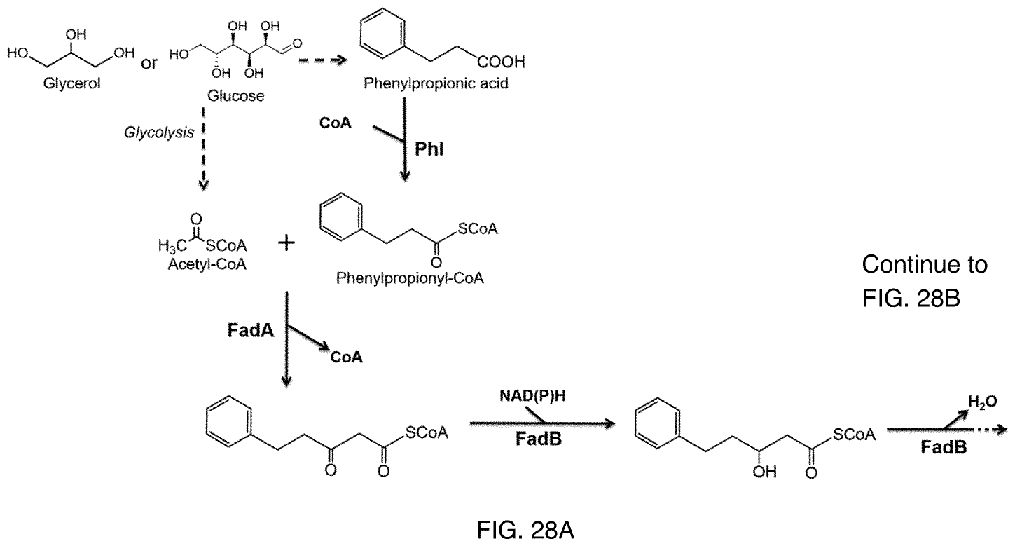

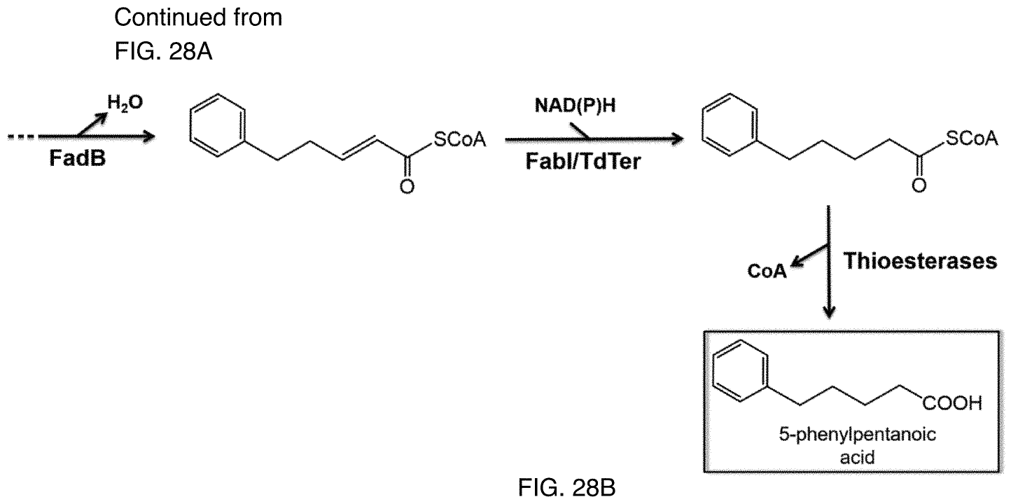

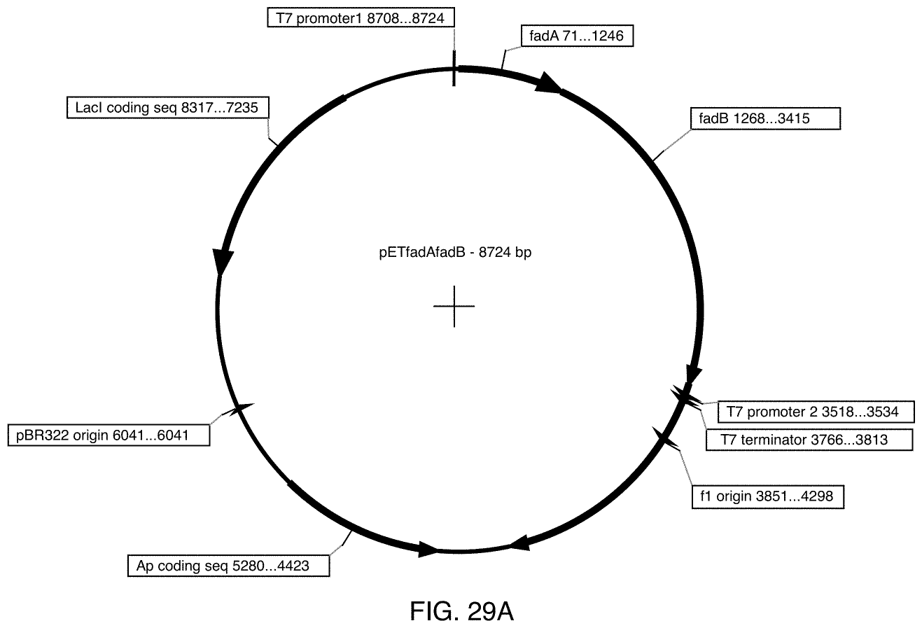

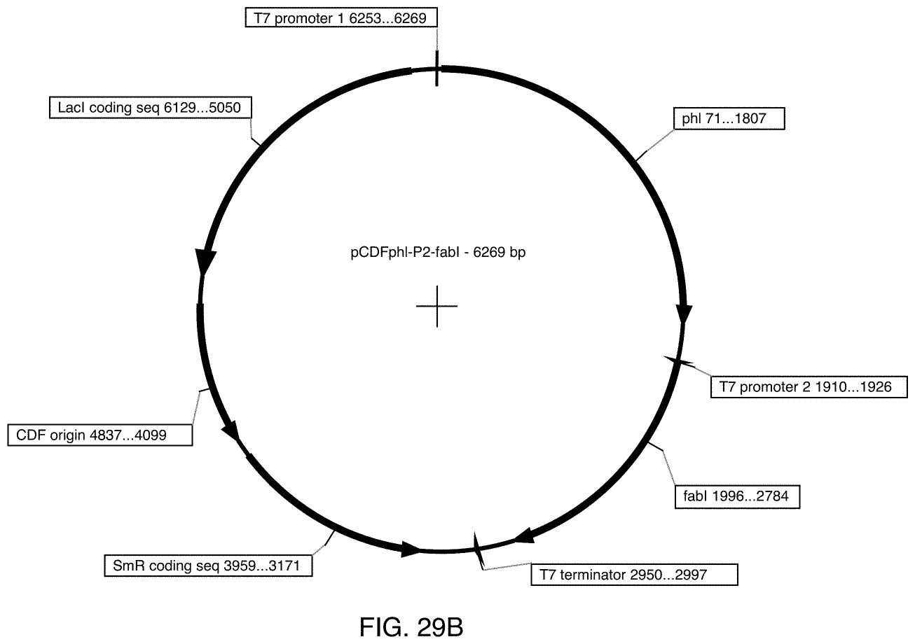

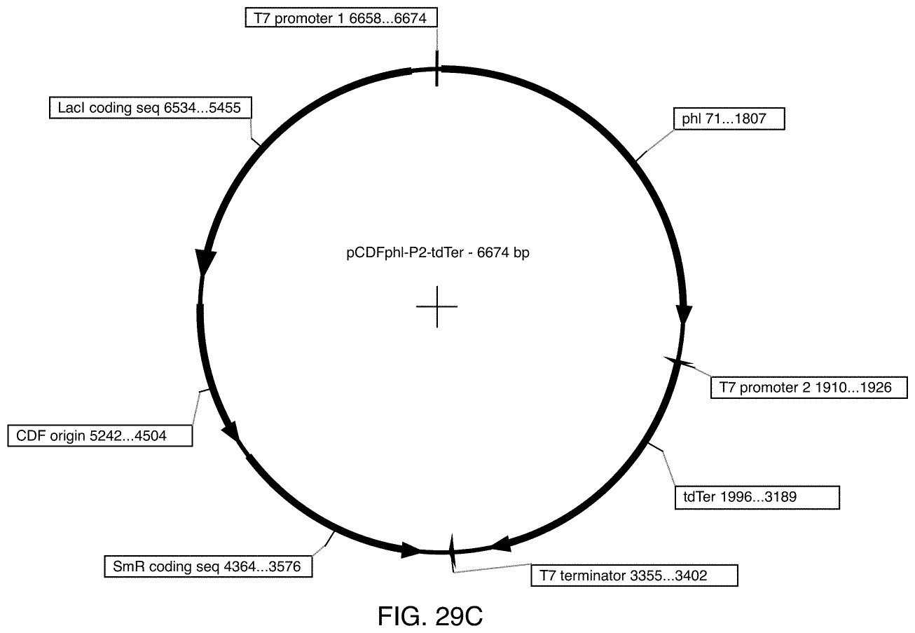

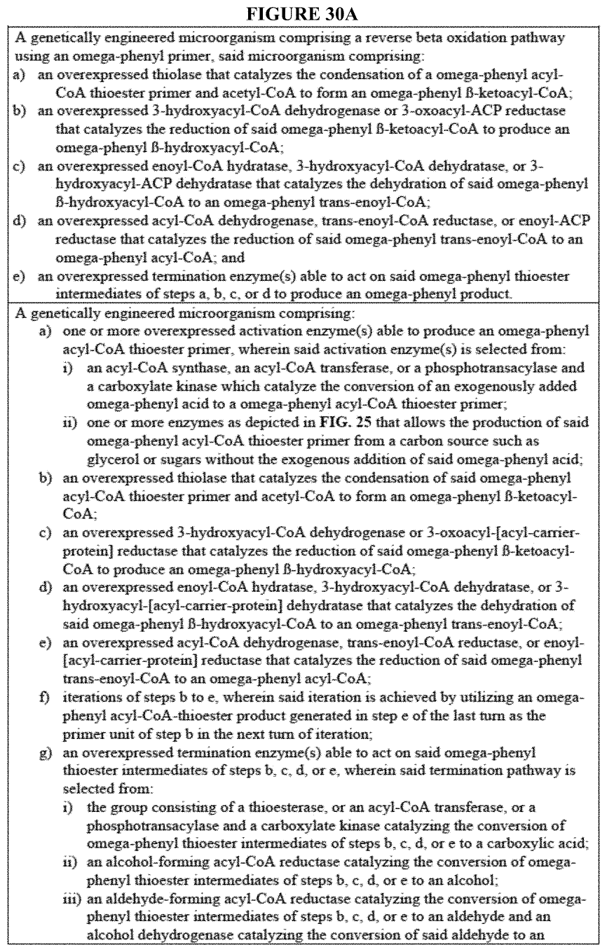

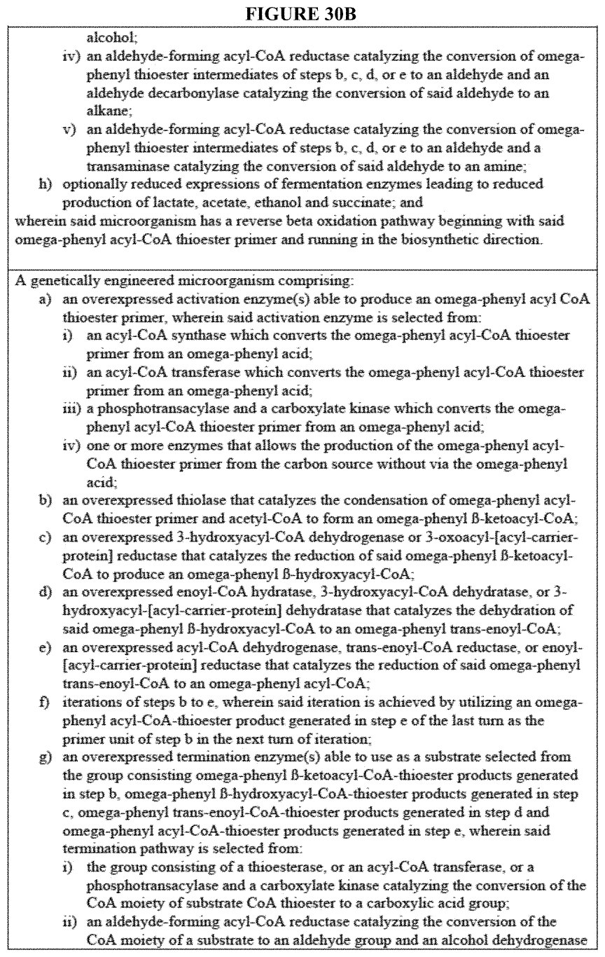

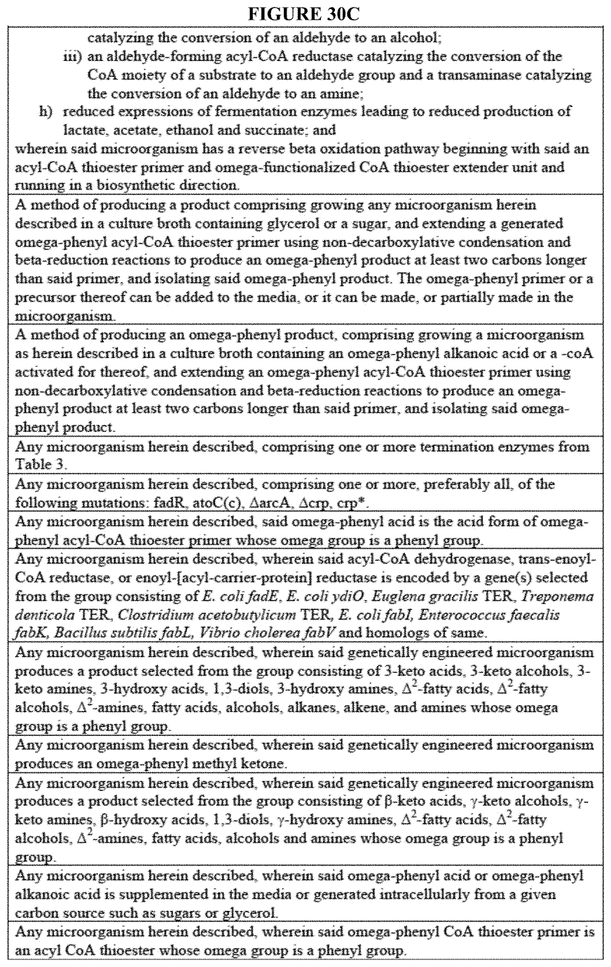

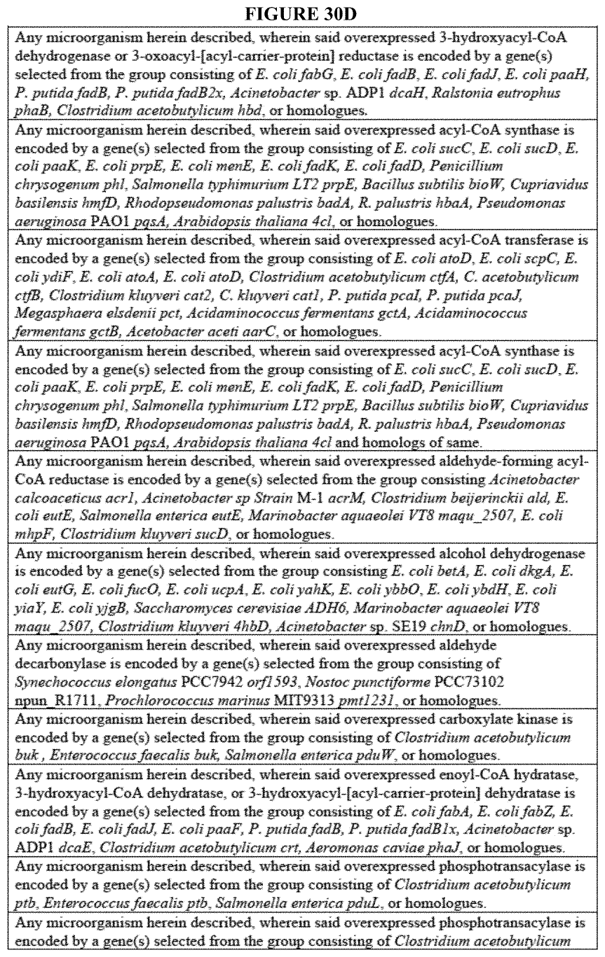

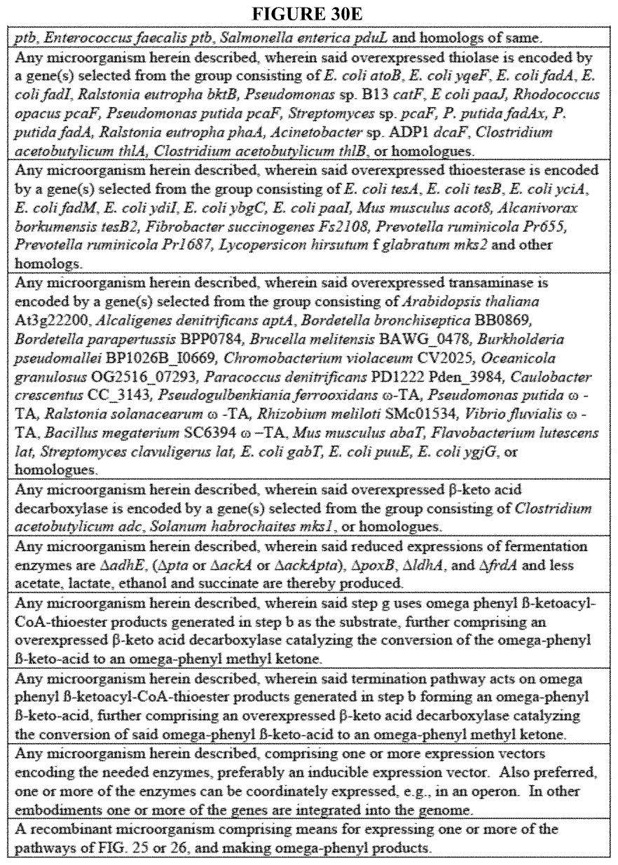

FIG. 2A-B (81): Synthesis of ω-1-carboxylated methyl ketones, 2-alcohols and 2-amines, namely ω-1 ketoacids, hydroxyacids and amino acids, through the platform depicted in FIG. 1 (R in FIG. 1=—COOH). Omega-carboxylated acyl-CoA, which is activated from α,ω-diacid, serves as the primer.

FIG. 3A-B (81): Derivatives of ω-1 ketoacids, hydroxyacids and amino acids, which could be synthesized through additional enzymatic and metabolic reactions. Products shown include omega-functionalized methyl ketones, 2-alcohols, 2-amines, and their derivatives, including lactams, lactones, α,ω-1-diamines, ω-1-amino-1-alcohols, ω-amino methyl ketones, ω-hydroxy methyl ketones, ω-amino-2-alcohols, α,ω-1-diols.

FIG. 4 (81): Example of synthesis of levulinic acid (4-oxopentanoic acid) through the proposed platform with succinyl-CoA as the primer. Succinyl-CoA is activated from succinate by Cat1. Levulinic acid is produced after subsequent condensation between succinyl-CoA and acetyl-CoA catalyzed by PaaJ, CoA removal catalyzed by PcaIJ and decarboxylation by Mks1/Adc.

FIG. 5 (81): Titers of levulinic acid synthesized through the platform depicted in FIG. 4 with different enzymes catalyzing the first four steps. JST06(DE3) ΔsdhB, an E. coli strain deficient of mixed-acid fermentations, thioesterases and TCA cycle, served as the host strain. The engineered strains were grown for 48 hours at 37° C. in 20 mL LB-like MOPS media supplemented with 20 g/L glycerol and 20 mM succinate.

FIG. 6A-B (81): The pathway to validate and demonstrate the iterative carbon elongation platform utilizing thiolase-catalyzed non-decarboxylative Claisen condensation which accepts omega-functionalized acyl-CoA primers. The validation is through analyzing whether omega-functionalized carboxylic acids or omega-functionalized alcohols are produced after adding termination pathways acyl-CoA thioesterase/transferase (ACT) or acyl-CoA reductase+alcohol dehydrogenase (ACR+ADH) respectively at the acyl-CoA node of the platform. Omega-functionalization was demonstrated (see FIG. 7-11): omega-phenylation (R=-Ph); omega-carboxylation (R=—COOH); omega-hydroxylation (R=—OH) and omega-1-methylation (R═—CH(CH₃)₂).

FIG. 7 (81): Titers of omega-phenylalkanoic acids produced with phenylacetyl-CoA (R=Phenyl) as the primer. Utilized host strain and enzymatic components are listed in the bottom part. “Endogenous” refers to native enzymes without overexpression. The engineered strain was grown for 48 hours at 30° C. in 20 mL LB-like MOPS media supplemented with 20 g/L glycerol and 5 mM phenylacetic acid.

FIG. 8 (81): Titers of dicarboxylic acids and omega-hydroxy acids produced with succinyl-CoA and glutaryl-CoA (R=—COOH) as the primer, and omega-1-methyl fatty acid and omega-1-methyl alcohol with isobutyryl-CoA (R=—CH(CH₃)₂) as the primer. Utilized host strain and enzymatic components are listed in the bottom part. “Endogenous” herein refers to native enzymes without overexpression. The engineered strains were grown for 48 hours at 37° C. (when using succinyl-CoA or glutaryl-CoA as the primer) or 30° C. (when using isobutyryl-CoA as the primer) in 20 mL LB-like MOPS media supplemented with 20 g/L glycerol and 20 mM succinate or glutaric acid or isobutyric acid.

FIG. 9 (81): Total ion GC-MS chromatogram showing peak of 4-hydroxybutyric acid synthesized with glycolyl-CoA (R=—OH) as the primer. The following enzymes provided the individual components of the pathway: BktB (thiolase) and PhaB1 (HACDH) from Ralstonia eutropha, Aeromonas caviae PhaJ (ECH), Treponema denticola TdTer (ECR) with native enzymes catalyzing the acid-forming termination and Megasphaera elsdenii transferase Pct activating glycolic acid to glycolyl-CoA. MG1655 (DE3) ΔglcD served as the host strain. The engineered strain was grown for 96 hours at 30° C. in 50 mL LB media supplemented with 10 g/L glucose and 40 mM glycolic acid.

FIG. 10 (81): Improvement of adipic acid synthesis and synthesis of dicarboxylic acids of different chain lengths through the iterative system depicted in FIG. 6 (81) with succinyl-CoA priming and specified pathway enzymes listed in the bottom part. The engineered strains were grown for 48 hours at 37° C. in 20 mL LB-like MOPS media supplemented with 20 g/L glycerol and 20 mM succinate.

FIG. 11 (81): Adipic acid production from glycerol through the pathway depicted in FIG. 6 priming from succinyl-CoA without the addition of primer precursor succinic acid in either shake flasks or controlled bioreactors.

FIG. 12A-B (81): Synthesis of omega-phenyl methyl ketones, 2-alcohols and 2-amines, through the platform depicted in FIG. 1(R=-Phenyl). Omega-phenylacyl-CoA, which is activated from omega-phenylalkanoic acid, serves as the primer.

FIG. 13A-B (81): Synthesis of omega-1-methyl methyl ketones, 2-alcohols and 2-amines, through the platform depicted in FIG. 1 (R=—CH(CH₃)₂). Omega-1-methyl acyl-CoA, which is activated from omega-1-methylated carboxylic acid, serves as the primer.

FIG. 14A-B (81): Synthesis of ω-hydroxy methyl ketones, α,ω-1-diols and ω-1-amino-1-alcohols, through the platform depicted in FIG. 1 (R=—OH). Omega-hydroxyacyl-CoA, which is activated from omega-hydroxyacid, serves as the primer.

FIG. 15A-B (81): Synthesis of ω-amino methyl ketones, ω-amino-2-alcohols and α,ω-1-diamines, through the platform depicted in FIG. 1 (R=—NH₂). Omega-amino acyl-CoA, which is activated from omega-amino acid, serves as the primer.

FIG. 16A-B (81): Synthesis of ω-halogenated methyl ketones, ω-halogenated 2-alcohols and ω-halogenated 2-amines, through the platform depicted in FIG. 1 (R=—X). Omega-halogenated acyl-CoA, which is activated from omega-halogenated carboxylic acid, serves as the primer.

FIG. 17 A-C (81): A partial listing of embodiments, any one or more or which can be combined with any other, even if not yet so combined.

FIG. 18A-B (1-84): Platform for the synthesis of omega-1-functionalized carboxylic acids, alcohols, amines, hydrocarbons, and methyl ketones. The platform is composed of thiolase, dehydrogenase, dehydratase and reductase. Thiolase(s) catalyzes the condensation between omega-1-functionalized primer and extender unit acetyl-CoA and generates omega-1-functionalized β-keto acyl-CoA. Dehydrogenase converts omega-1-functionalized β-keto acyl-CoA to omega-1-functionalized β-hydroxy acyl-CoA. Dehydratase converts omega-1-functionalized β-hydroxy acyl-CoA to omega-1-functionalized enoyl-CoA. Reductase converts omega-1-functionalized enoyl-CoA to omega-1-functionalized acyl-CoA. The platform can be iterated by using synthesized omega-1-functionalized acyl-CoA as the primer for the next turn of the platform.

Termination pathways starting from four omega-1-functionalized CoA thioester intermediates terminate the platform and generate various omega-1-functionalized carboxylic acids, alcohols and amines with different β-reduction degrees. There are four types of termination pathways: 1) thioesterase/CoA-transferase/phosphotransacylase+kinase which generates carboxylic acids; 2) alcohol-forming acyl-CoA reductase or aldehyde-forming acyl-CoA reductase and alcohol dehydrogenase which generates alcohols; 3) aldehyde-forming acyl-CoA reductase and aldehyde decarbonylase which generates hydrocarbons (not pictured); and 4) aldehyde-forming acyl-CoA reductase and transaminase which generates amines.

Secondary termination pathways are also possible. For example, omega-1-functionalized methyl ketone can be generated by subsequent decarboxylation of omega-1-functionalized β-keto acid. Omega-1-functionalized acyl-CoA thioester primers be generated from their acid form, which can be either supplemented in the media or derived from other carbon sources, or directly synthesized through additional cellular pathways.

R means functionalized group of primers, intermediates and products. n means length of primers, intermediates and products. Dashed line means multiple reaction steps or iteration.

FIG. 19A-B (2-84): Proposed platform and its products utilizing omega-1-methyl acyl-CoA as the primer (R=—CH3).

FIG. 20A-B (3-84): Example pathway of synthesis of 4-methylpentanoic acid and 4-methylpentanol through the proposed platform with isobutyryl-CoA as the primer and acetyl-CoA as the extender unit. Isobutyryl-CoA is activated by Pct from isobutyric acid. The platform is composed of thiolase BktB, which catalyzes the condensation between primer isobutyryl-CoA and extender unit acetyl-CoA to form 4-methyl-3-oxopentanoyl-CoA; dehydrogenase and dehydratase FadB, which catalyzes the conversion of 4-methyl-3-oxopentanoyl-CoA to 4-methyl-3-hydroxypentanoyl-CoA and the subsequent dehydration of 4-methyl-3-hydroxypentanoyl-CoA to 4-methyl-2-pentenoyl-CoA; reductase Fab1, which reduces 4-methyl-2-pentenoyl-CoA to 4-methylpentanoyl-CoA. Termination reaction by endogenous thioesterases or overexpressed YdiI converts 4-methylpentanoyl-CoA to the product 4-methylpentanoic acid. Acyl-CoA reductase and alcohol dehydrogenase Maqu_2507 terminates the platform and catalyzes the termination reaction of reduction of 4-methylpentanoyl-CoA to 4-methylpentanal and the subsequent reduction of 4-methylpentanal to the product 4-methylpentanol.

FIG. 21A-B (6-84): Proposed platform and its products utilizing omega-1-amino acyl-CoA as the primer (R=—NH2).

FIG. 22A-B (7-84): Proposed platform and its products utilizing omega-1-hydroxy acyl-CoA as the primer (R=—OH).

FIG. 23A (8a-84) Derivatization reaction of omega-1 amino acid, one of the products of the platform depicted in FIG. 21A-B, to lactam, catalyzed by amidohydrolase.

FIG. 23B (8b-84) Derivatization reaction of omega-1 hydroxy acid, one of the products of the platform depicted in FIG. 22A-B, to lactone, catalyzed by lactonase.

FIG. 24 A-G (9-84) A partial listing of preferred embodiments, and one or more of which can be combined with any other one or more shown here.

FIG. 25A-B (2-85): Example pathways for the generation of omega-phenyl acyl-CoA thioester primers benzoyl-CoA, phenylacetyl-CoA and phenylpropionyl-CoA from carbon sources such as glucose or glycerol via chorismate, the intermediate of biosynthesis of aromatic amino acids phenylalanine and tryptophan.

FIG. 26A-B (3-85): Example pathway of synthesis of 4-phenylbutyric acid and 6-phenylhexanoic acid through the proposed platform with phenylacetyl-CoA as the primer and acetyl-CoA as the extender unit. Phenylacetyl-CoA is activated by E. coli enzyme PaaK from phenylacetic acid. The platform is composed of thiolase FadA from Pseudomonas putida, which catalyzes the condensation between primer phenylacetyl-CoA and extender unit acetyl-CoA to 4-phenylacetoacetyl-CoA; dehydrogenase and reductase FadB from P. putida, which catalyzes the conversion of 4-phenylacetoacetyl-CoA to 4-phenyl-3-hydroxybutyryl-CoA and the subsequent dehydration of 4-phenyl-3-hydroxybutyryl-CoA to 4-phenylcrotonyl-CoA; reductase Fab1 from E. coli or Ter from Treponema denticola (tdTER), which reduces 4-phenylcrotonyl-CoA to 4-phenylbutyryl-CoA. Termination by an acid forming reaction, such as those catalyzed by thioesterases, can convert the intermediate of one-turn of the pathway, 4-phenylbutyryl-CoA, to the product 4-phenylbutyric acid. Pathway iteration using the generated 4-phenylbutyryl-CoA as a primer with similar thiolase, dehydrogenase, dehydratase and reductase steps results in 6-phenylhexonyl-CoA, which can be converted to 6-phenylhexanoic acid through acid forming termination pathways.

FIG. 27A-C(6-85): Maps of vectors overexpressing required enzymes for the production of even chain omega-phenyl products, such as 4-phenylbutyric acid and 6-phenylhexanoic acid, through the proposed platform depicted in FIG. 26A-B with phenylacetyl-CoA as the primer.

FIG. 28A-B (7-85): Example pathway of synthesis of 5-phenylpentanoic acid through the proposed platform with phenylpropionyl-CoA as the primer and acetyl-CoA as the extender unit. Phenylpropionyl-CoA is activated by Penicillium chrysogenum enzyme Phl from phenylpropionic acid. The platform is composed of thiolase FadA from Pseudomonas putida, which catalyzes the condensation between primer phenylpropionyl-CoA and extender unit acetyl-CoA to 5-phenyl-3-oxopentanoyl-CoA; dehydrogenase and reductase FadB from P. putida, which catalyzes the conversion of 5-phenyl-3-oxopentanoyl-CoA to 5-phenyl-3-hydroxypentanoyl-CoA and the subsequent dehydration of 5-phenyl-3-hydroxypentanoyl-CoA to 5-phenyl-2-pentenoyl-CoA; reductase Fab1 from E. coli or TdTer, which reduces 5-phenyl-2-pentenoyl-CoA to 5-phenylpentanoyl-CoA. Termination by an acid forming reaction, such as those catalyzed by thioesterases, converts 5-phenylpentanoyl-CoA to the product 5-phenylpentanoic acid.

FIG. 29A-C (8-85): Maps of vectors overexpressing enzymes for the production of odd chain omega-phenyl products, such as 5-phenylpentanoic acid, through the proposed platform depicted in FIG. 28A-B with phenylpropionyl-CoA as the primer.

FIG. 30A-E (9-85): A partial listing of preferred embodiments, and one or more of which can be combined with any other one or more.

[symbol]

TABLE 1 ACTIVATION ENZYMES

TABLE 2 REACTIONS OF THE PLATFORM

TABLE 3 PRIMARY & SECONDARY TERMINATION ENZYMES

TABLE 4 STRAINS & PLASMIDS

TABLE 5 OLIGONUCLEOTIDES

TABLE 6 HOST STRAINS AND PLASMIDS ENABLING OMEGA-FUNCTIONALIZED SMALL MOLECULE SYNTHESIS WITH LISTED PRIMER/EXTENDER UNIT COMBINATIONS

### DETAILED DESCRIPTION

The disclosure generally relates to the use of microorganisms to make omega- and omega-1-functionalized products. The method entails developing a new pathway that is based on native or engineered thiolases capable of catalyzing the condensation of omega-functionalized acyl-CoA primers with an acetyl-CoA as the extender unit. This has been reported in neither the scientific, peer-reviewed literature nor the patent literature.

The first enzyme needed in the new pathway are activation enzymes. TABLE 1 lists several activation enzymes. Once the functionalized initiating primer is ready, it must be condensed with another Acetyl-CoA by a thiolase. Thiolases that will work with these functionalized primers are listed in TABLE 2. The remaining reactions in the platform tend to be less fussy about substrates, so many known enzymes will work with functionalized intermediates. These are also listed in TABLE 2. TABLE 3 shows various termination pathways, including both primary pathways and secondary pathways, and exemplary enzymes that can be used therein.

The following description provides additional details, any one of which can be subject to patenting in combination with any other. The specification in its entirety is to be treated as providing a variety of details that can be used interchangeably with other details, as the specification would be of inordinate length if one were to list every possible combination of genes/vectors/enzymes/hosts that can be made to enable carbon source conversion into omega- or omega-1-functionalized products.

### Methods

Initial demonstration of the engineered pathway was conducted in E. coli for convenience. Enzymes of interest where expressed from vectors such as pETDuet-1 or pCDFDuet-1 (MERCK, Germany), which makes use of the DE3 expression system. Genes can be codon optimized according to the codon usage frequencies of the host organism and synthesized by a commercial vendor or in-house. However, thousands of expression vectors and hosts are available, and this is a matter of convenience.

Pathway enzymes can also be inserted into the host chromosome, allowing for the maintenance of the pathway without requiring antibiotics to ensure the continued upkeep of plasmids. A large number of genes that can be placed on the chromosome, as chromosomal expression does not require separate origins of replication as is the case with plasmid expression.

Engineered strains expressing pathway components can be cultured under the following or similar conditions. Overnight cultures started from a single colony can be used to inoculate flasks containing appropriate media. Cultures are grown for a set period of time, and the culture media analyzed. The conditions will be highly dependent on the specifications of the actual pathway and what exactly is to be tested. For example, the ability for the pathway to be used for omega-1-functionalized product synthesis can be tested by the glycerol or sugars as a substrate in MOPS minimal media, as described by Neidhardt et al (1974), supplemented with appropriate antibiotics, and inducers. Depending on the strain chosen, primers or precursors for primers can be added to the medium, or they can be internally generated.

Wild-type K12 Escherichia coli strain MG1655 was used as the host for all genetic modifications. All resulting strains used in this study are listed in TABLE 4. Gene deletions were performed using P1 phage transduction with single-gene knockout mutants from the National BioResource Project (NIG, Japan) as the specific deletion donor. The λDE3 prophage, carrying the T7 RNA polymerase gene and lacIq, was integrated into the chromosome through λDE3 lysogenization kit (Novagen, Darmstadt, Germany). All strains were stored in 32.5% glycerol stocks at −80° C. Plates were prepared using LB medium containing 1.5% agar, and appropriate antibiotics were included at the following concentrations: ampicillin (100 m/mL), spectinomycin (50 μg/mL), kanamycin (50 μg/mL), and chloramphenicol (34 m/mL).

All plasmids used in this study and oligonucleotides used in their construction are listed in TABLE 5 and TABLE 6. Plasmid based gene overexpression was achieved by cloning the desired gene(s) into either pETDuet-1 or pCDFDuet-1 (Novagen, Darmstadt, Germany) digested with appropriate restriction enzymes using In-Fusion PCR cloning technology (Clontech Laboratories, Inc., Mountain View, Calif.). Cloning inserts were created via PCR of ORFs of interest from their respective genomic or codon-optimized DNA with Phusion polymerase (Thermo Scientific, Waltham, Mass.) E. coli genes were obtained from genomic DNA, while heterologous genes were synthesized by GenScript (Piscataway, N.J.) or GeneArt (Life Technologies, Carlsbad, Calif.) with codon optimization except for bktB, phaB 1, pct, cbjALD and mks1, which were amplified from genomic DNA or cDNA of their source organisms. The recognition site of NdeI in the paaH sequence was eliminated via overlap PCR. The resulting In-Fusion products were used to transform E. coli Stellar cells (Clontech Laboratories, Inc., Mountain View, Calif.) and PCR identified clones were confirmed by DNA sequencing.

The minimal medium designed by Neidhardt et al. with 125 mM MOPS and Na₂HPO₄ in place of K₂HPO₄ (1.48 mM for fermentations in flasks; 2.8 mM for fermentations in bioreactors), supplemented with 20 g/L glycerol, 10 g/L tryptone, 5 g/L yeast extract, 100 μM FeSO₄, 5 mM calcium pantothenate, 5 mM (NH₄)₂SO₄, and 30 mM NH₄Cl was used for all fermentations unless otherwise stated.

Neutralized 5 mM phenylacetic acid or 20 mM succinic acid, glutaric acid, isobutyric acid, glycolic acid, or propionic acid was supplemented as needed. Antibiotics (50 μg/mL carbenicillin and 50 μg/mL spectinomycin) were included when appropriate. All chemicals were obtained from Fisher Scientific Co. (Pittsburgh, Pa.) and Sigma-Aldrich Co. (St. Louis, Mo.).

Unless otherwise stated, fermentations were performed in 25 mL Pyrex Erlenmeyer flasks (narrow mouth/heavy duty rim, Corning Inc., Corning, N.Y.) filled with 20 mL fermentation medium and sealed with foam plugs filling the necks. A single colony of the desired strain was cultivated overnight (14-16 h) in LB medium with appropriate antibiotics and used as the inoculum (1%). After inoculation, flasks were incubated in a NBS 124 Benchtop Incubator Shaker (New Brunswick Scientific Co., Inc., Edison, N.J.) at 200 rpm and 37° C., except fermentations supplemented with phenylacetic acid or isobutyric acid in which the temperature was 30° C. When optical density (550 nm, OD₅₅₀) reached ˜0.3-0.5, 5 μM isopropyl β-D-1-thiogalactopyranoside (IPTG) was added for plasmid based gene expression in all cases except the following: 1 μM IPTG was used for adipic acid production from glycerol without succinic acid supplementation and 10 μM IPTG was used during production of ω-phenylalkanoic acids. For induction of controlled chromosomal expression constructs, 0.1 mM cumate and 15 ng/mL anhydrotetracycline were also added when appropriate. Flasks were then incubated under the same conditions for 48 h post-induction unless otherwise stated.

Additional fermentations were conducted in a SixFors multi-fermentation system (Infors HT, Bottmingen, Switzerland) with an air flow rate of 2 N L/hr, independent control of temperature (37° C.), pH (controlled at 7.0 with NaOH and H₂SO₄), and stirrer speed (660 rpm for adipic acid production and 720 rpm for tiglic acid production). Fermentations for adipic acid production used the above fermentation media with 45 g/L glycerol, the inclusion of 5 μM sodium selenite, and 1 μM IPTG. Pre-cultures were grown in 25 mL Pyrex Erlenmeyer flasks as described above and incubated for 24 h post-induction. An appropriate amount of this pre-culture was centrifuged, washed twice with fresh media, and used for inoculation (400 mL initial volume).

Fermentations with glycolyl-CoA as a primer were conducted in 250 mL Erlenmeyer Flasks filled with 50 mL LB media supplemented with 10 g/L glucose and appropriate antibiotics. The cultivation of inoculum was same as above but 2% inoculation was used. After inoculation, cells were cultivated at 30° C. and 250 rpm in a NBS 124 Benchtop Incubator Shaker until an optical density of ˜0.8 was reached, at which point IPTG (0.1 mM) and neutralized glycolic acid (40 mM) were added. Flasks were then incubated under the same conditions for 96 h for production of 4-hydroxybutyric acid.

For analysis of dicarboxylic acids and ω-hydroxy acids, extractions were performed as previously described (Clomburg et al. 2015), with 12-hydroxydodecanoic acid as the internal standard and diethyl ether as the organic solvent. With the exception of 4-methylpentanol analysis, extraction of all other analysis samples was conducted as previously described (Kim et al. 2015), with tridecanoic acid as the internal standard and hexane:MTBE (1:1) as the organic solvent.

Extracted products were then derivatized by BSTFA (N,O-bis(trimethylsilyl)trifluoroacetamide) as previously described (Clomburg et al. 2015) for GC-MS or GC-FID analysis. For GC-FID analysis of 4-methylpentanol, extraction was performed with hexane:MTBE as described above with tridecanol as the additional internal standard. Acetylation was then conducted by adding a 1:1 pyridine:acetic anhydride mixture, following the previously described method (Kim et al. 2015). For GC-MS analysis of 4-methylpentanol, samples were extracted with hexane, with 1-heptanol as the internal standard, with subsequent BSTFA derivatization

GC-MS metabolite identification: Except for identifications of 4-hydroxybutyric acid, metabolite identification was conducted via GC-MS as previously described in an Agilent 7890A GC system (Agilent Technologies, Santa Clara, Calif.), equipped with a 5975C inert XL mass selective detector (Agilent) and Rxi-5Sil column (0.25 mm internal diameter, 0.10 μm film thickness, 30 m length; Restek, Bellefonte, Pa.). The sample injection amount was 2 μL with 40:1 split ratio. The injector and detector were maintained at 280° C. The column temperature was held initially at 35° C. for 1 min and increased to 200° C. at the rate of 6° C./min, then to 270° C. at the rate of 30° C./min. That final temperature was maintained for 1 min before cooling back to initial temperature. The carrier gas was helium (2.6 mL/min, Matheson Tri-Gas, Longmont, Colo.).

Identification of 4-hydroxbutyric acid was conducted by the Baylor College of Medicine Analyte Center (www.bcm.edu/research/centers/analyte, Houston, Tex.). An Agilent 6890 GC system (Agilent Technologies, Santa Clara, Calif.), equipped with a 5973 mass selective detector (Agilent Technologies) and HP-5 ms column (Agilent Technologies) was used. Sample extraction was conducted using Agilent Chem Elut liquid extraction columns (Agilent Technologies) according to manufacturer protocols.

Product quantification was conducted using previously reported gas chromatography methods. Quantification was performed in Varian CP-3800 gas chromatograph (Varian Associates, Inc., Palo Alto, Calif.), equipped with a flame ionization detector (GC-FID) and an Agilent HP-5 capillary column (0.32 mm internal diameter, 0.50 μm film thickness, 30 m length. Agilent). The temperature was initially 50° C., held for 3 min, then increased to 250° C. at 10° C./min, and finally 250° C. was held for 10 min. Helium (1.8 mL/min, Matheson Tri-Gas) was used as the carrier gas. The injector and detector temperatures were 220 and 275° C., respectively. The sample was injected at 1 μL without splits.

The concentration of glycerol, adipic acid, 6-hydroxyhexanoic acid, 7-hydroxyheptanoic acid and 4-methylpentanoic acid were determined via ion-exclusion HPLC using a Shimadzu Prominence SIL 20 system (Shimadzu Scientific Instruments, Inc., Columbia, Md.) equipped with an HPX-87H organic acid column (Bio-Rad, Hercules, Calif.) with operating conditions to optimize peak separation (0.3 ml/min flow rate, 30 mM H₂SO₄ mobile phase, column temperature 42° C.).

### Omega Functionalized Products

We first validated the iterative operation of the proposed carbon chain elongation platform consisting of thiolase accepting various ω-functionalized primers, along with HACD, ECH and ECR, and achieved synthesis of various ω-functionalized carboxylic acids and alcohols after termination at the acyl-CoA node by ACT and ACR+ADH respectively (FIG. 6A-B).

The aromatic primer phenylacetyl-CoA, with acetyl-CoA as the extender unit, was used to achieve iterative pathway operation and synthesis of corresponding aromatic products. Pseudomonas putida thiolase FadA (ppFadA) was used, with P. putida FadB (ppFadB) providing HACD and ECH activities, Escherichia coli Fab1 as the ECR, and E. coli acyl-CoA synthetase PaaK to activate externally supplied phenylacetic acid.

These and subsequent enzymes for all of the pathways described herein were selected on the basis of literature reports of the specific enzymes' and organisms' ability to function with the required intermediates. When expressed in mixed-acid fermentation-deficient E. coli MG1655 ΔldhAΔpoxBΔptaΔadhEΔfrdA (JC01), these enzymes enabled the synthesis of 4-phenylbutyric acid (177 mg/L) and 6-phenylhexanoic acid (49 mg/L) (FIG. 7). These products result from the action of endogenous termination pathways, possibly native ACTs, on acyl-CoA's that are generated by one and two turns of the pathway, respectively.

Omega-carboxylated primers can support the synthesis of products such as ω-hydroxyacids and dicarboxylic acids. In this context, we selected succinyl-CoA and glutaryl-CoA, which can be generated from corresponding acids by the Clostridium kluyveri CoA transferase Cat1. Overexpression of E. coli PaaJ (thiolase), PaaH (HACD), and PaaF (ECH), with Treponema denticola trans-enoyl-CoA reductase (TdTer) as the ECR in JC01 led to production of C6 (adipic, 170 mg/L) and C7 (pimelic, 25 mg/L) dicarboxylic acids from endogenous acid-producing termination enzymes following succinic or glutaric acid supplementation, respectively (FIG. 8).

The system's modularity was exploited to achieve the synthesis of ω-hydroxyacids by manipulation of termination pathways. Minimizing activity of endogenous acid-producing termination reactions (by deletion of native thioesterases) and using Clostridium beijerinckii ACR cbjALD (with native ADH enzymes) in combination with the other pathway components enabled the synthesis of 6-hydroxyhexanoic acid (34 mg/L) and 7-hydroxyheptanoic acid (87 mg/L) following supplementation with exogenous succinic or glutaric acid, respectively (FIG. 8). This strategy used the thioesterase-deficient strain JST06 (JC01 ΔyciAΔybgCΔydiIΔtesAΔfadMΔtesB), as ω-hydroxyacids were not observed when JC01 was used as the host strain. This demonstrates the importance of engineering the termination pathway(s) for product selectivity, and it represents an area in which further optimization could improve target product synthesis and reduce byproduct formation via nonspecific and/or endogenous enzymes.

Usage of ω-hydroxylated primer glycolyl-CoA can lead to the synthesis of ω-hydroxyacid 4-hydroxybutyric acid through the proposed pathway (FIG. 6A-B, FIG. 9). The following enzymes provided the individual components of the pathway: BktB (thiolase) and PhaB1 (HACD) from Ralstonia eutropha, Aeromonas caviae PhaJ (ECH), Treponema denticola TdTer (ECR) with native enzymes catalyzing the acid-forming termination and Megasphaera elsdenii transferase Pct activating glycolic acid to glycolyl-CoA. MG1655 (DE3) ΔglcD served as the host strain.

The use of functionalized primers and termination pathways enables the synthesis of a wide range of products, albeit at relatively low titers. One potential cause of low product titers is the intracellular concentrations of primers and/or extender units available for condensation. To determine whether low primer concentrations affected product synthesis, we attempted to maximize succinyl-CoA availability by deleting sdhB (encoding a subunit of succinate dehydrogenase), thereby reducing succinate consumption through the tricarboxylic acid (TCA) cycle. This deletion was introduced into JST06 to reduce undesirable hydrolysis of priming (succinyl-CoA) and extending units (acetyl-CoA) by native thioesterases, with Mus musculus dicarboxylic ACT Acot8 then overexpressed as the termination enzyme. This re-engineered strain produced a higher adipic acid titer (334 mg/L compared to 170 mg/L in the JC01 background) in the presence of succinic acid (FIG. 10).

Further product diversification from the use of succinyl-CoA can be achieved through iterative pathway operation. Replacement of the thiolase (PaaJ), HACD (PaaH) and ECH (PaaF) pathway components with the Acinetobacter sp. ADP1 enzymes DcaF, DcaH, and DcaE resulted in the production of suberic (34 mg/L) and sebacic (13 mg/L) acids in addition to adipic acid (95 mg/L) (FIG. 10). These C8 and C10 diacids, products of two and three turns of the pathway, respectively, were not observed when using PaaJHF, demonstrating how selecting individual pathway components with desired specificity can control product synthesis. This type of approach could be used to further increase product diversity, as well as overall performance, through the selection and engineering of enzymes with required specificity and efficiency for desired functionalization.

Although our system can synthesize functionalized products, primer precursor supplementation and low overall titers need to be overcome to achieve industrial scale viability. To show the potential for higher product titer from a single carbon source, improvement in adipic acid production was targeted, given the industrial importance of this compound. The intracellular generation of succinic acid/succinyl-CoA was accomplished using strain MG1655 ΔldhAΔpoxBΔptaΔadhE (MB263), which retains the reductive branch of the TCA cycle, along with the overexpression of PaaJ, PaaH, PaaF, TdTer, Cat1, and Acot8, resulting in 0.24 g/L adipic acid from a single carbon source (glycerol, FIG. 11). Maximization of primer availability through deletion of sucD, which encodes a subunit of succinyl-CoA synthetase, part of the TCA cycle, was again used to improve product titer (0.35 g/L, FIG. 11). When grown in a controlled bioreactor with a higher initial glycerol concentration, this strain produced 2.5 g/L adipic acid (4.1% mol/mol glycerol) (FIG. 11). Further improvement is envisioned through minimizing acetate formed directly through the transferase for primer activation. Acetate recycling (to acetyl-CoA) or use of an acetyl-CoA-independent activation enzyme offers a potential solution to improve adipic acid titer, a strategy that can also be applied to other combinations of primer and extenders.

Once we demonstrated the iterative operation of the proposed platform and its acceptance of various ω-functionalized primers, we then utilized this platform to demonstrate the synthesis of ω-functionalized methyl ketone. We chose ω-carboxylated succinyl-CoA as the primer, and ω-carboxylated methyl ketone levulinic acid, the product from first cycle of β-ketoacyl-CoA node and a key building block for the chemical industry. Levulinic acid production was observed in JST06 ΔsdhB strain overexpressing PaaJ, and Cat1 along with P. putida CoA transferase PcaIJ which generates 3-oxoadipic acid from 3-oxoadipyl-CoA, the product of condensation between succinyl-CoA and acetyl-CoA (48 mg/L) (FIG. 5). 3-oxoadipic acid was believed to be spontaneously decarboxylated to levulinic acid in this strain. Additional overexpression of the decarboxylases Solanum habrochaites Mks1 or Clostridium acetobutylicum Adc increased levulinic acid titers to 71 mg/L and 159 mg/L, respectively (FIG. 5). All the strains were grown with glycerol and succinic acid for the synthesis of levulinic acid.

### Omega-1 Products

For these experiments, isobutyrate (precursor for the initiating omega-1 methyl (—CH₃) primer) was added to a concentration of 20 mM in the medium. Isobutyryl-CoA priming (FIG. 20A-B) was assessed with the following individual pathway components: Megasphaera elsdenii Pct (transferase for isobutyric acid activation), Ralstonia eutropha BktB (thiolase), E. coli FadB (HADCH and ECH), and Euglena gracilis EgTer (ECR). Overexpression of these enzymes in mixed-acid fermentation-deficient E. coli MG1655 ΔldhA ΔpoxB Δpta ΔadhE ΔfrdA (JC01), enabled the synthesis of 4-methylpentanoic acid (FIG. 8). This product, representing a one-turn reversal with isobutyryl-CoA priming, resulted from endogenous termination pathways. Overexpression of E. coli YdiI (thioesterase) resulted in slight increases to 4-methylpentanoic acid titer (FIG. 8), indicting the value of overexpressing termination enzymes.

The use of different termination pathways enables the production of products with varying functionality, even when exploiting the same initial omega-1-functionalzied primer. For example, engineering termination pathways through replacing YdiI with the Marinobacter aquaeolei alcohol-forming acyl-CoA reductase Maqu2507, along with the use of host strain with deletion to native thioesterases (JC01 ΔyciA ΔybgC ΔydiI ΔtesA ΔfadM ΔtesB ΔfadE), enabled production of 4-methylpentanol (FIG. 8). Whereas the production of 4-methylpentanoic acid results from an acid forming termination pathways from 4-methylpentanoyl-CoA, 4-methylpentanol is the result of the 2-step reduction of this omega-1-methylated intermediate to form the corresponding alcohol. As such, the modular nature of the engineered pathway provides the opportunity to produce a wide range of products through the combinatorial engineering of the primers and termination pathway utilized.

Iterative pathway operation using primers such as omega-1-methyl- (FIG. 19A-B), omega-1-amino- (FIG. 21A-B)), and omega-1-hydroxyl-acyl-CoA thioesters (FIG. 22A-B) as the initiating primer, in combination with various termination pathways) enables the engineered pathway to synthesize various omega-1-functionalized carboxylic acids, alcohols, hydrocarbons, and amines with different degrees of β-reduction and carbon chain length as described herein.

### Omega Phenyl Products

Generation of the required omega-phenyl acyl-CoA thioester primer can make use of externally supplied phenylalkanoic acids or -CoA form thereof or can be accomplished from a carbon source such as glycerol or sugars through the pathways depicted in FIG. 25A-B. Exploiting components of pathways for the biosynthesis of aromatic amino acids phenylalanine and tryptophan, the generation of omega-phenyl acyl-CoA thioester primers benzoyl-CoA, phenylacetyl-CoA and phenylpropionyl-CoA can be accomplished via chorismate, enabling the synthesis of required omega-phenyl primers, and subsequent omega-phenyl products, from industrially relevant single carbon sources such as sugars or glycerol.

Combining the core engineered pathway with enzymes/pathways for the generation of the initial omega-phenyl acyl-CoA thioester primer provides a route for the generation of omega-phenyl acyl-CoA intermediates with varying beta-functionality. These intermediates can then be converted to numerous products of interest through action of various termination pathways. For example, the use of acid forming termination pathways, such as thioesterases, enables the synthesis of omega-phenyl carboxylic acids, while alcohol forming termination pathways, such as acyl-CoA reductases/alcohol dehydrogenases, provides a route to various omega-phenyl alcohols. The combinatorial expression of core pathway components with termination pathways allows the synthesis of omega-phenyl products, including omega-phenyl carboxylic acids, alcohols, hydrocarbons, amines, methyl ketones and their beta-functionalized derivatives.

Initial demonstration of the engineered pathway was conducted in E. coli for convenience, and focused on the synthesis of omega-phenyl carboxylic acids. Enzymes of interest where expressed from vectors such as pETDuet-1 or pCDFDuet-1 (MERCK, Germany), which makes use of the DE3 expression system. Genes can be codon optimized according to the codon usage frequencies of the host organism and synthesized by a commercial vendor or in-house. However, thousands of expression vectors and hosts are available, and this is a matter of convenience. The vectors used in initial demonstration of the engineered pathway are shown in FIG. 27A-C and FIG. 29A-C.

For these experiments, phenylacetate or phenylpropionate was added to the growth medium at a concentration of 5 mM to provide the starting substrate. The aromatic primer phenylacetyl-CoA, with acetyl-CoA as the extender unit, was used to achieve pathway operation and demonstrate the synthesis of phenylalkanoic acids.

Pseudomonas putida thiolase FadA, P. putida FadB (providing both HACD and ECH activities) was tested with either E. coli Fab1 or T. denticola TER as the ECR, and E. coli acyl-CoA synthetase PaaK to activate externally supplied phenylacetic acid. Overexpression of either combination of enzymes in mixed-acid fermentation-deficient E. coli MG1655 ΔldhA ΔpoxB Δpta ΔadhE ΔfrdA (JC01), enabled the synthesis of 4-phenylbutyric acid (FIG. 7). This product resulted from the action of endogenous termination pathways, possibly native thioesterases, on phenylbutyryl-CoA generated by one turn of the pathway. In addition to demonstrating overall pathway functionality, the use of either T. denticola TER or E. coli Fab1 with FadA and FadB for 4-phenylbutyric acid synthesis also demonstrates how both β-oxidation enzymes and fatty acid biosynthesis enzymes acting on the required CoA intermediates can be used in this context.

Iterative pathway operation (e.g. the use of the omega-phenyl acyl-CoA generated from a turn of the pathway as a primer for the next round) was also demonstrated through the use of P. putida thiolase FadA, P. putida FadB (providing HACD and ECH activities), E. coli Fab1 (ECR), and E. coli acyl-CoA synthetase PaaK in the JC01 strain background. Varying induction levels by altering IPTG concentration (10 μM) as well as incubation at 30° C., resulted in the synthesis of 6-phenylhexanoic acid in addition to higher levels of 4-phenylbutyric acid, compared to the above results with the same set of enzymes (FIG. 7). This demonstrates the ability to synthesize omega-phenyl products of various chain length through the iterative addition of 2 carbon units (via acetyl-CoA as the donor) to the growing omega-phenyl acyl-CoA primer.

Combination of iterative pathway operation using any of benzoyl-CoA (FIG. 25A-B, phenylacetyl-CoA (FIG. 25A-B and FIG. 26A-B) and phenylpropionyl-CoA (FIG. 25A-B) and FIG. 28A-B) as the initial primer with various termination pathways enables the engineered pathway to synthesize various omega-phenyl carboxylic acids, alcohols, hydrocarbons, and amines with different degrees of β-reduction and carbon chain length as described herein.

In addition, pathway and process optimization, in line with industrial biotechnology approaches, can improve performance for a specific target product, as the underlying carbon and energy efficiency enables the feasibility of further advancing product titer, rate, and yield. Important areas of optimization include generating and balancing pools of priming and extender units and optimization of required pathway enzymes for a given target product. The former can exploit previously developed pathways for primers and extender units, whereas the latter includes identifying and engineering enzymes that may be flux limiting due to suboptimal enzyme specificity or activity. These approaches will be continually aided by developments in protein and metabolic engineering and synthetic and systems biology.

### Other Species

The above experiments are repeated in Bacillus subtilis. The same genes can be used, especially since Bacillus has no significant codon bias. A protease-deficient strain like WB800N is preferably used for greater stability of heterologous protein. The E. coli-B. subtilis shuttle vector pMTLBS72 exhibiting full structural stability can be used to move the genes easily to a more suitable vector for Bacillus. Alternatively, two vectors pHT01 and pHT43 allow high-level expression of recombinant proteins within the cytoplasm. As yet another alternative, plasmids using the theta-mode of replication such as those derived from the natural plasmids pAMβ1 and pBS72 can be used. Several other suitable expression systems are available. Since the FAS genes are ubiquitous, the invention is predicted to function in Bacillus.

The above experiments are repeated in yeast. The same genes can be used, but it may be preferred to accommodate codon bias. Several yeast E. coli shuttle vectors are available for ease of the experiments. Since the FAS genes are ubiquitous, the invention is predicted to function in yeast, especially since yeasts are already available with exogenous functional TE genes and the reverse beta-oxidation pathway has also been made to run in yeast.

Each of the following is incorporated by reference herein in its entirety for all purposes:
61/440,192, filed Feb. 7, 2011, WO2012109176, filed Feb. 7, 2012, and US20130316413 Reverse beta-oxidation pathway
62/140,628, Mar. 31, 2015, WO2017020043 Biosynthesis of polyketides
61/932,057, filed Jan. 27, 2014, WO2015112988, US20160340699, TYPE II FATTY ACID SYNTHESIS ENZYMES IN REVERSE beta-OXIDATION.
62/069,850, filed Oct. 29, 2014, WO2016069929, SYNTHETIC PATHWAY FOR BIOSYNTHESIS FROM 1-CARBON COMPOUNDS
61/531,911, filed Sep. 7, 2011; 61/440,192, filed Feb. 7, 2011; WO2013036812, US20140273110 Functionalized carboxylic acids and alcohols by reverse fatty acid oxidation
62/011,465, Filed Jun. 12, 2014; WO2015191972, WO2015191972, Omega-hydroxylated carboxylic acids
62/012,113, filed Jun. 13, 2014; WO2015191422A1, WO2015191972, WO2016007258, Omega-aminated carboxylic acids
62/011,474, filed Jun. 12, 2014; WO2015191422A1, WO2015191972 Omega-carboxylated carboxylic acids and derivatives
62/154,397, filed Apr. 29, 2015, WO2016176347, SYNTHESIS OF OMEGA-1 FUNCTIONALIZED PRODUCTS AND DERIVATIVES THEREOF (herein referred to as “81”);
62/148,248, filed Apr. 16, 2015, WO2016168708, SYNTHESIS OF OMEGA FUNCTIONALIZED METHYLKETONES, 2-ALCOHOLS, 2-AMINES, AND DERIVATIVES THEREOF (herein referred to as “84”); and
62/154,010, filed Apr. 28, 2015, WO2016176339, SYNTHESIS OF OMEGA-PHENYL PRODUCTS AND DERIVATIVES THEREOF (herein referred to as “85”)
Cheong, S., et al., Energy- and carbon-efficient synthesis of functionalized small molecules in bacteria using non-decarboxylative Claisen condensation reactions. Nat. Biotechnol. 34 (5) (2016).
Choi, K. H., et al., β-Ketoacyl-Acyl Carrier Protein Synthase III (FabH) Is a Determining Factor in Branched-Chain Fatty Acid Biosynthesis. J. Bacteriol. 182, 365-370 (2000).
Clomburg, J. M. et al. Integrated engineering of β-oxidation reversal and ω-oxidation pathways for the synthesis of medium chain ω-functionalized carboxylic acids. Metab. Eng. 28, 202-212 (2015).
Clomburg, J. M., et al., A Synthetic Biology Approach to Engineer a Functional Reversal of the β-Oxidation Cycle. ACS Synthetic Biology 1, 541-554 (2012).
Dellomonaco, C., et al., Engineered reversal of the β-oxidation cycle for the synthesis of fuels and chemicals. Nature 476, 355-359 (2011).
Haapalainen, A. M., et al., The thiolase superfamily: condensing enzymes with diverse reaction specificities. Trends in Biochemical Sciences 31, 64-71 (2006).
Heath, R. J. & Rock, C. O. The Claisen condensation in biology. Nat. Prod. Rep. 19, 581-596 (2002).
Jiang, C., et al., Divergent evolution of the thiolase superfamily and chalcone synthase family. Molecular Phylogenetics and Evolution 49, 691-701 (2008).
Kim, S., Clomburg, J. M. & Gonzalez, R. Synthesis of medium-chain length (C6-C10) fuels and chemicals via β-oxidation reversal in Escherichia coli. J. Ind. Microbiol. Biotechnol. 42, 465-475 (2015).
Lan, E. I., et al., Metabolic engineering of 2-pentanone synthesis in Escherichia coli. Aiche J. 59, 3167-3175 (2013).
Lian J. & Zhao, H., Reversal of the β-Oxidation Cycle in Saccharomyces cerevisiae for Production of Fuels and Chemicals, ACS SYN. BIOL. 4, 332-341 (2015).
Neidhardt, F. C., et al., Culture medium for enterobacteria. J. Bacteriol. 119, 736-747 (1974).
Pfleger, B. F., et al., Metabolic engineering strategies for microbial synthesis of oleochemicals. Metab. Eng. 29, 1-11 (2015).
Vick, J. E. et al. Escherichia coli enoyl-acyl carrier protein reductase (Fab1) supports efficient operation of a functional reversal of the β-oxidation cycle. Appl. Environ. Microbiol. 81, 1406-1416 (2015).

The following claims are provided to add additional clarity to this disclosure. Future applications claiming priority to this application may or may not include the following claims, and may include claims broader, narrower, or entirely different from the following claims. Furthermore, any detail from any claim may be combined with any other detail from another claim, even if not yet so combined.

1) A genetically engineered microorganism, comprising:
a) an overexpressed activation enzyme(s) able to produce an omega-1-(ω-1) functionalized CoA thioester primer, wherein said activation enzyme is selected from:
i) an acyl-CoA synthase that generates an ω-1-functionalized CoA thioester primer from an ω-1-functionalized acid;
ii) an acyl-CoA transferase that generates the ω-1-functionalized CoA thioester primer from an ω-1-functionalized acid;
iii) a phosphotransacylase and a carboxylate kinase that generates the ω-1-functionalized CoA thioester primer from an ω-1-functionalized acid;
iv) one or more enzymes that generates the ω-1-functionalized CoA thioester primer from a carbon source without proceeding via an ω-1-functionalized acid;
b) an overexpressed thiolase that catalyzes the condensation of said ω-1-functionalized acyl-CoA primer with acetyl-CoA to form an ω-1-functionalized beta-ketoacyl-CoA;
c) an overexpressed 3-hydroxyacyl-CoA dehydrogenase or 3-oxoacyl-[ACP] reductase that catalyzes the reduction of said ω-1-functionalized beta-ketoacyl-CoA to produce an ω-1-functionalized beta-hydroxyacyl-CoA;
d) an overexpressed enoyl-CoA hydratase, 3-hydroxyacyl-CoA dehydratase, or 3-hydroxyacyl-[ACP] dehydratase that catalyzes the dehydration of said ω-1-functionalized beta-hydroxyacyl-CoA to an ω-1-functionalized trans-enoyl-CoA;
e) an overexpressed acyl-CoA dehydrogenase, trans-enoyl-CoA reductase, or enoyl-[ACP] reductase that catalyzes the reduction of said ω-1-functionalized trans-enoyl-CoA to an ω-1-functionalized acyl-CoA;
f) an overexpressed termination enzyme(s) able to remove ω-1-intermediates from steps b-e and produce an ω-1-functionalized product;
g) optionally reduced expression of fermentation genes leading to reduced production of lactate, acetate, ethanol and succinate; and
h) wherein said microorganism has a reverse beta-oxidation pathway beginning with said ω-1-functionalized CoA thioester primer and running in a biosynthetic direction.

2) The microorganism of claim 1, wherein said ω-1-functionalized CoA thioester primer is functionalized with a group selected from alkyl group, hydroxyl group, carboxyl group, aryl group, halogen, amino group, hydroxyacyl group, carboxyacyl group, aminoacyl group, ketoacyl group, halogenated acyl group, and a functionalized acyl group.

3) The microorganism of claim 1, wherein said termination pathway comprises one or more of:
a) a thioesterase, or an acyl-CoA transferase, or a phosphotransacylase and a carboxylate kinase catalyzing a conversion of an omega-1-functionalized thioester intermediate of steps b, c, d, or e to a carboxylic acid;
b) an alcohol-forming acyl-CoA reductase catalyzing a conversion of said omega-1-functionalized intermediates of steps b, c, d, or e to an alcohol;
c) an aldehyde-forming acyl-CoA reductase catalyzing a conversion of said omega-1-functionalized thioester intermediates of steps b, c, d, or e to an aldehyde, and an alcohol dehydrogenase catalyzing a conversion of said aldehyde to an alcohol;
d) an aldehyde-forming acyl-CoA reductase catalyzing a conversion of said omega-1-functionalized thioester intermediates of steps b, c, d, or e to an aldehyde, and an aldehyde decarbonylase catalyzing a conversion of said aldehyde to an alkane; or
e) an aldehyde-forming acyl-CoA reductase catalyzing a conversion of said omega-1-functionalized thioester intermediates of steps b, c, d, or e to an aldehyde, and a transaminase catalyzing a conversion of said aldehyde to an amine;
f) an overexpressed β-keto acid decarboxylase catalyzing a conversion of an omega-1-functionalized ß-keto-acid to an omega-1-functionalized methyl ketone;
g) an overexpressed amidohydrolase catalyzing a conversion of an omega-1 amino acid to a lactam; or
h) an overexpressed lactonase catalyzing the conversion of an omega-1 hydroxy acid to a lactone.

4) The microorganism of claim 1, wherein said activation enzyme is encoded by a gene(s) selected from E. coli paaK; E. coli sucCD; E. coli fadK; E. coli fadD; E. coli prpE; E. coli menE; Penicillium chrysogenum phl; Salmonella typhimurium LT2 prpE; Bacillus subtilis bioW; Cupriavidus basilensis hmfD; Rhodopseudomonas palustris badA; R. palustris hbaA; Pseudomonas aeruginosa PAO1 pqsA; Arabidopsis thaliana 4cl; E. coli atoD; E. coli atoA; E. coli scpC; Clostridium kluyveri cat1; Clostridium kluyveri cat2; Clostridium acetobutylicum ctfAB; Pseudomonas putida pcaIJ; Megasphaera elsdenii pct; Acidaminococcus fermentans gctAB; Acetobacter aceti aarC; E. coli ydiF; Clostridium acetobutylicum ptb; Enterococcus faecalis ptb; Salmonella enterica pduL; Clostridium acetobutylicum buk, Enterococcus faecalis buk and Salmonella enterica pduW.

5) The microorganism of claim 1, wherein said overexpressed thiolase is encoded by a gene(s) selected from the group consisting of E. coli atoB, E. coli yqeF, E. coli fadA, E. coli fadI, Ralstonia eutropha bktB, Pseudomonas sp. B13 catF, E coli paaJ, Rhodococcus opacus pcaF, Pseudomonas putida pcaF, Streptomyces sp. pcaF, P. putida fadAx, P. putida fadA, Ralstonia eutropha phaA, Acinetobacter sp. ADP1 dcaF, Clostridium acetobutylicum thlA, and Clostridium acetobutylicum thlB.

6) The microorganism of claim 1, wherein said overexpressed 3-hydroxyacyl-CoA dehydrogenase or 3-oxoacyl-[acyl-carrier-protein] reductase is encoded by a gene(s) selected from the group consisting of E. coli fabG, E. coli fadB, E. coli fadB, E. coli paaH, P. putida fadB, P. putida fadB2x, Acinetobacter sp. ADP1 dcaH, Ralstonia eutrophus phaB, and Clostridium acetobutylicum hbd.

7) The microorganism of claim 1, wherein said overexpressed enoyl-CoA hydratase, 3-hydroxyacyl-CoA dehydratase, or 3-hydroxyacyl-[acyl-carrier-protein] dehydratase is encoded by a gene(s) selected from the group consisting of E. coli fabA, E. coli fabZ, E. coli fadB, E. coli fadJ, E. coli paaF, P. putida fadB, P. putida fadBlx, Acinetobacter sp. ADP1 dcaE, Clostridium acetobutylicum crt, and Aeromonas caviae phaJ.

8) The microorganism of claim 1, wherein said acyl-CoA dehydrogenase, trans-enoyl-CoA reductase, or enoyl-[acyl-carrier-protein] reductase is encoded by a gene(s) selected from the group consisting of E. coli fadE, E. coli ydiO, Euglena gracilis TER, Treponema denticola TER, Clostridium acetobutylicum TER, E. coli fabI, Enterococcus faecalis fabK, Bacillus subtilis fabL, and Vibrio cholerea fabV.

9) The microorganism of claim 1, wherein said termination enzymes are selected from one or more enzymes encoded by a gene(s) selected from E. coli tesA; E. coli tesB; E. coli yciA; E. coli fadM; E. coli ydiI; E. coli ybgC; E. coli paal; Mus musculus acot8; Lycopersicon hirsutum f glabratum mks2; Alcanivorax borkumensis tesB2; Fibrobacter succinogenes Fs2108; Prevotella ruminicola Pr655; Prevotella ruminicola Pr1687; E. coli atoD; E. coli atoA; E. coli scpC; Clostridium kluyveri cat1; Clostridium kluyveri cat2; Clostridium acetobutylicum ctfAB; Pseudomonas putida pcaIJ; Megasphaera elsdenii pct; Acidaminococcus fermentans gctAB; Acetobacter aceti aarC; E. coli ydiF; Clostridium acetobutylicum ptb; Enterococcus faecalis ptb; Salmonella enterica pduL; Clostridium acetobutylicum buk, Enterococcus faecalis buk, Salmonella enterica pduW; Lycopersicon hirsutum f glabratum mks1; Clostridium acetobutylicum adc; Arabidopsis thaliana At3g22200; Alcaligenes denitrificans AptA; Bordetella bronchiseptica BB0869; Bordetella parapertussis BPP0784; Brucella melitensis BAWG_0478; Burkholderia pseudomallei BP1026B_I0669; Chromobacterium violaceum CV2025; Oceanicola granulosus OG2516_07293; Paracoccus denitrificans PD1222 Pden_3984; Pseudogulbenkiania ferrooxidans ω-TA; Pseudomonas putida ω-TA; Ralstonia solanacearum ω-TA; Rhizobium meliloti SMc01534; Vibrio fluvialis ω-TA; Mus musculus abaT; Flavobacterium lutescens lat; Streptomyces clavuligerus lat; E. coli gabT; E. coli puuE; E. coli ygjG; Clostridium beijerinckii adh; E. coli sera; Gordonia sp. TY-5 adh1; Gordonia sp. TY-5 adh2; Gordonia sp. TY-5 adh3; Rhodococcus ruber adh-A; Acidaminococcus fermentans hgdH; Comamonas testosteroni pmdD; Xanthomonas campestris XCC1745; Homo sapiens PON1; Mesorhizobium loti Mlr6805; Pseudomonas sp. P51 tcbE; Flavobacterium sp. KI72 nylB; Arthrobacter sp. KI72 nylA; Homo sapiens DPYS; Brevibacillus agri pydB; E. coli pyrC; Pseudomonas putida crnA; Pseudomonas fluorescens puuE; Acinetobacter calcoaceticus acr1; Acinetobacter sp Strain M-1 acrM; Clostridium beijerinckii ald; E. coli eutE; Salmonella enterica eutE; E. coli mhpF; Clostridium kluyveri sucD; E. coli betA; E. coli dkgA; E. coli eutG; E. coli fucO; E. coli ucpA; E. coli yahK; E. coli ybbO; E. coli ybdH; E. coli yiaY; E. coli yjgB; Saccharomyces cerevisiae ADH6; Clostridium kluyveri 4hbD; Acinetobacter sp. SE19 chnD; Arabidopsis thaliana At3g22200; Alcaligenes denitrificans AptA; Bordetella bronchiseptica BB0869; Bordetella parapertussis BPP0784; Brucella melitensis BAWG_0478; Burkholderia pseudomallei BP1026B_I0669; Chromobacterium violaceum CV2025; Oceanicola granulosus OG2516_07293; Paracoccus denitrificans PD1222 Pden_3984; Pseudogulbenkiania ferrooxidans ω-TA; Pseudomonas putida ω-TA; Ralstonia solanacearum ω-TA; Rhizobium meliloti SMc01534; Vibrio fluvialis ω-TA; Mus musculus abaT; Flavobacterium lutescens lat; Streptomyces clavuligerus lat; E. coli gabT; E. coli ygjG; and E. coli puuE.

10) The microorganism of claim 1, wherein said reduced expression of fermentation enzymes are ΔadhE, (Δpta or ΔackA or ΔackApta), ΔpoxB, ΔldhA, and ΔfrdA.

11) The microorganism of claim 1, comprising the following mutations: fadR, atoC(c), ΔarcA, Δcrp, crp*.

12) A recombinant microorganism, said microorganism having a reverse beta oxidation pathway running in a biosynthetic direction and comprising overexpressed enzymes including 1) a thiolase catalyzing a non-decarboxylative Claisen condensation between an ω-1-functionalized primer and acetyl-CoA, 2) a hydroxyacyl-CoA dehydrogenase, 3) an enoyl-CoA hydratase, 4) an enoyl-CoA reductase and 5) a termination enzyme(s) catalyzing conversion of intermediates of said reverse beta oxidation pathway to one or more ω-1-functionalized product(s).

13) The recombinant microorganism of claim 12, said microorganism being a bacteria.

14) The recombinant microorganism of claim 12, said microorganism being E. coli.

15) A method of making ω-1-functionalized products, comprising growing a microorganism of claim 1 in a nutrient broth under conditions such that said enzymes are overexpressed, said microorganism producing ω-functionalized product using said overexpressed enzymes, and isolating said ω-functionalized product or a derivative of said ω-functionalized product.

16) The method of claim 15, said nutrient broth being supplemented with an ω-1-functionalized acid.

17) The method of claim 15, wherein said microorganism is a microorganism of claim 3.

18) The method of claim 15, wherein said microorganism a microorganism of claim 4.

19) The method of claim 16, wherein said microorganism a microorganism of claim 4.

20) The method of claim 16, wherein said microorganism a microorganism of claim 9.
